SEQUENCE LISTING (4 sequences)

SEQ ID NO: 1 - protein, 5 aa, Artificial Sequence
  Synthetic peptide
AKERC

SEQ ID NO: 2 - protein, 5 aa, Artificial Sequence
  Synthetic peptide
CREKA

SEQ ID NO: 3 - protein, 8 aa, Artificial Sequence
  Synthetic peptide
ARYLQKLN

SEQ ID NO: 4 - protein, 8 aa, Artificial Sequence
  Synthetic peptide
AXYLXXLN

(19) United States
(12) Patent Application Publication
(10) Pub. No.: US 2017/0151181 A1
(43) Pub. Date: Jun. 1, 2017

(54) METHODS OF TREATING CANCERS WITH THERAPEUTIC NANOPARTICLES

(71) Applicant: PFIZER INC., New York, NY (US)

(72) Inventor: Jason Summa, Cambridge, MA (US)

(73) Assignee: Pfizer Inc., New York, NY (US)

(21) Appl. No.: 15/294,061

(22) PCT Filed: Apr. 17, 2015
(86) PCT No.: PCT/US2015/026498

§ 371 Date: Oct. 14, 2016

Related U.S. Application Data
(60) Provisional application No. 61/981,327, filed on Apr. 18, 2014.
(60) Provisional application No. 61/988,474, filed on May 5, 2014.
(60) Provisional application No. 62/080,124, filed on Nov. 14, 2014.
(60) Provisional application No. 62/081,836, filed on Nov. 19, 2014.

(51) Int. Cl. A61K 9/51; A61K 9/00; A61K 31/337

(52) U.S. Cl. CPC A61K 9/5161; A61K 31/337; A61K 9/0019; A61K 9/5153

(57) ABSTRACT

The present disclosure relates in part to methods of treating cancers having a mutation in a Ras gene in a patient in need thereof, comprising administering to the patient a therapeutically effective amount of a nanoparticle composition, wherein the nanoparticle composition comprises nanoparticles.

### CROSS-REFERENCE TO RELATED APPLICATIONS

This application claims the benefit of and priority to U.S. Provisional Patent Application No. 62/081,836, filed Nov. 19, 2014, U.S. Provisional Patent Application No. 62/080,124, filed Nov. 14, 2014, U.S. Provisional Patent Application No. 61/988,474, filed May 5, 2014, and U.S. Provisional Patent Application No. 61/981,327, filed Apr. 18, 2014, each of which is hereby incorporated by reference in its entirety.

### BACKGROUND

Systems that deliver certain drugs to a patient (e.g., targeted to a particular tissue or cell type or targeted to a specific diseased tissue but not normal tissue), or that control release of drugs has long been recognized as beneficial.

For example, therapeutics that include an active drug and that are e.g., targeted to a particular tissue or cell type or targeted to a specific diseased tissue but not to normal tissue, may reduce the amount of the drug in tissues of the body that are not targeted. This is particularly important when treating a condition such as cancer where it is desirable that a cytotoxic dose of the drug is delivered to cancer cells without killing the surrounding non-cancerous tissue. Effective drug targeting may reduce the undesirable and sometimes life threatening side effects common in anticancer therapy. In addition, such therapeutics may allow drugs to reach certain tissues they would otherwise be unable to reach.

Therapeutics that offer controlled release and/or targeted therapy also must be able to deliver an effective amount of drug, which is a known limitation in other nanoparticle delivery systems. For example, it can be a challenge to prepare nanoparticle systems that have an appropriate amount of drug associated each nanoparticle, while keeping the size of the nanoparticles small enough to have advantageous delivery properties. However, while it is desirable to load a nanoparticle with a high quantity of therapeutic agent, nanoparticle preparations that use a drug load that is too high will result in nanoparticles that are too large for practical therapeutic use.

Accordingly, a need exists for nanoparticle therapeutics and methods of making such nanoparticles, that are capable of delivering therapeutic levels of drug to treat diseases such as cancer, while also reducing patient side effects.

### SUMMARY

Described herein are methods of treating cancers having a mutation in a Ras gene in a patient in need thereof, comprising administering to the patient a therapeutically effective amount of a nanoparticle composition, wherein the nanoparticle composition comprises nanoparticles.

In one aspect, a method of treating cancer having a Ras mutation in a patient in need thereof is provided. The method comprises administering to the patient a therapeutically effective amount of a nanoparticle composition comprising anti-cancer nanoparticles.

In certain embodiments, the Ras mutation is a mutation in a Ras gene selected from the group consisting H-Ras, K-Ras, and N-Ras. For example, in certain embodiments, the Ras gene is K-Ras.

In certain embodiments, the therapeutically effective amount of the nanoparticle composition is about 50 to about 75 mg/m² of the therapeutic agent, or about 60 to about 75 mg/m² of the therapeutic agent, or about 60 mg/m² of the therapeutic agent.

In certain embodiments, the therapeutic agent is docetaxel.

In certain embodiments, contemplated methods further comprise administering a contemplated nanoparticle composition about every three weeks to said patient. In certain embodiments, a contemplated nanoparticle composition is administered by intravenous infusion over about 1 hour.

In certain embodiments, the cancer treated by contemplated methods was not stabilized by administration to the patient of free therapeutic agent.

In certain embodiments, the hydrodynamic diameter of contemplated nanoparticles is about 60 to about 150 nm, or about 90 to about 140 nm, or about 90 to about 120 nm.

In certain embodiments, the contemplated nanoparticles substantially retain the therapeutic agent for at least 1 minute when placed in a phosphate buffer solution at 37° C. In certain embodiments, contemplated nanoparticles substantially immediately release less than about 30% of the therapeutic agent when placed in a phosphate buffer solution at 37° C. In certain embodiments, contemplated nanoparticles release about 10 to about 45% of the therapeutic agent over about 1 hour when placed in a phosphate buffer solution at 37° C.

In certain embodiments, contemplated nanoparticles comprise a diblock poly(lactic) acid-poly(ethylene) glycol copolymer or a diblock poly(lactic acid-co-glycolic acid)-poly(ethylene)glycol copolymer. For example, in certain embodiments, the poly(lactic) acid-poly(ethylene)glycol copolymer has a poly(lactic) acid number average molecular weight fraction of about 0.6 to about 0.95, or about 0.6 to about 0.8, or about 0.75 to about 0.85, or about 0.7 to about 0.9.

In certain embodiments, contemplated nanoparticles comprise about 10 to about 25 weight percent poly(ethylene)glycol, or about 10 to about 20 weight percent poly(ethylene)glycol, or about 15 to about 25 weight percent poly(ethylene)glycol, or about 20 to about 30 weight percent poly(ethylene)glycol.

In certain embodiments, contemplated nanoparticles further comprise about 0.2 to about 30 weight percent poly(lactic) acid-poly(ethylene)glycol copolymer functionalized with a targeting ligand. In certain embodiments, contemplated nanoparticles further comprise about 0.2 to about 30 weight percent poly(lactic) acid-co-poly(glycolic) acid-poly(ethylene)glycol copolymer functionalized with a targeting ligand. In certain embodiments, the targeting ligand is covalently bound to the poly(ethylene)glycol.

In certain embodiments, the cancer treated by contemplated methods is lung cancer. For example, in certain embodiments, the lung cancer is small cell lung cancer.

In another aspect, a method of treating a refractory cancer in a patient in need thereof, comprising administering to the patient a therapeutically effective amount of a nanoparticle composition comprising anti-cancer nanoparticles, wherein the refractory cancer is refractory to chemotherapy and/or radiation therapy alone.

In certain embodiments, the refractory cancer is lung cancer.

In certain embodiments, the refractory cancer is an adenocarcinoma selected from lung, colon, and pancreatic cancer; follicular thyroid cancer; undifferentiated thyroid cancer; myelodysplastic syndromes; and acute myeloid leukemia.

In certain embodiments, the therapeutic agent is a taxane, a vinca alkaloid, or an mTor inhibitor. For example, in certain embodiments, the therapeutic agent is docetaxel. In certain embodiments, contemplated nanoparticles comprise about 10 to about 20 weight percent of docetaxel.

In certain embodiments, a patient treated by a contemplated method has previously been administered another chemotherapeutic agent and/or radiation.

In certain embodiments, the therapeutically effective amount of a contemplated nanoparticle composition is about 50 to about 75 mg/m² of docetaxel, or about 60 to about 75 mg/m² of docetaxel, or about 60 mg/m² of docetaxel.

In yet another aspect, a method of treating cancer having a Ras mutation in a patient in need thereof is provided. The method comprises identifying the patient on the basis that the patient has a mutation in a Ras gene, and administering to the patient a therapeutically effective amount of a nanoparticle composition comprising anti-cancer nanoparticles.

In certain embodiments, identifying the patient comprises obtaining a sample from the patient, and subjecting the sample to a diagnostic assay thereby to determine the presence or absence of a Ras mutation.

In certain embodiments, the diagnostic assay comprises polymerase chain reaction and DNA sequencing.

In certain embodiments, the Ras mutation is a K-Ras mutation.

In certain embodiments, the therapeutic agent is docetaxel.

In certain embodiments, the polymer is a diblock poly(lactic) acid-poly(ethylene) glycol copolymer.

In certain embodiments, contemplated anti-cancer nanoparticles are selected from the group consisting of polymeric nanoparticles, inorganic nanoparticles, metal nanoparticles, ceramic nanoparticles, nanocrystals, liposomes, micelles, and dendrimers.

In certain embodiments, contemplated anti-cancer nanoparticles comprise a polymer.

In certain embodiments, contemplated anti-cancer nanoparticles comprise a therapeutic agent.

In certain embodiments, contemplated anti-cancer nanoparticles are formed from a therapeutic agent.

In certain embodiments, contemplated anti-cancer nanoparticles comprise a cyclodextrin.

In still another aspect, a method of treating cancer having a Ras mutation in a patient in need thereof is provided. The method comprises administering to the patient a therapeutically effective amount of a nanoparticle composition, wherein the nanoparticle composition comprises nanoparticles comprising a polymer and a therapeutic agent.

In certain embodiments, a contemplated nanoparticle composition is administered according to a treatment cycle. In certain embodiments, the treatment cycle is 1-30 days in length, 15-25 days in length, or 21 days in length.

In certain embodiments, the treatment cycle is repeated.

In certain embodiments, a contemplated method comprises 1-15 treatment cycles, 2-8 treatment cycles, or 4 treatment cycles.

In certain embodiments, a contemplated nanoparticle composition is administered once per treatment cycle.

In yet another aspect, a composition for use in the treatment of cancer having a Ras mutation in a patient in need thereof is provided, wherein the composition comprises anti-cancer nanoparticles.

In still another aspect, a composition for use in the treatment of a refractory cancer in a patient in need thereof is provided, wherein the refractory cancer is refractory to chemotherapy and/or radiation therapy alone, and wherein the composition comprises anti-cancer nanoparticles.

In yet another aspect, a composition for use in the treatment of cancer having a Ras mutation in a patient in need thereof is provided, wherein the patient is identified on the basis that the patient has a mutation in a Ras gene, and wherein the composition comprises anti-cancer nanoparticles.

In still another aspect, a composition for use in the treatment of cancer having a Ras mutation in a patient in need thereof is provided, wherein the composition comprises nanoparticles comprising a polymer and a therapeutic agent.

### BRIEF DESCRIPTION OF THE DRAWINGS

FIG. 1 is flow chart for an emulsion process for forming a disclosed nanoparticle, according to an embodiment.

FIGS. 2A and 2B show flow diagrams for a disclosed emulsion process, according to an embodiment.

FIG. 3 is a bar graph showing tumor response for all patients treated with a contemplated nanoparticle composition, according to an embodiment.

FIG. 4 is a bar graph showing tumor response for K-Ras mutant patients treated with a contemplated nanoparticle composition, according to an embodiment.

FIG. 5 is a plot showing relative change in tumor size as a function of time, according to an embodiment.

FIG. 6 is a plot showing progression free survival as a function of time for all patients as compared to K-Ras mutant patients, according to an embodiment.

FIG. 7 is a plot showing overall free survival as a function of time for all patients as compared to K-Ras mutant patients, according to an embodiment.

### DETAILED DESCRIPTION

Described herein are methods of treating cancers having a mutation in a Ras gene in a patient in need thereof, comprising administering to the patient a therapeutically effective amount of a nanoparticle composition, wherein the nanoparticle composition comprises nanoparticles.

Without wishing to be bound by any theory, it is believed that Ras-mutant tumors may be more vascularized as compared to Ras-normal tumors and that as a result therapeutic nanoparticles can accumulate and deliver a therapeutic agent more readily in the Ras-mutant tumors. Alternatively or additionally, and again without wishing to be bound by any theory, it is believed that Ras-mutant tumors may endocytose nanoparticles more readily as compared to Ras-normal tumors, thereby resulting in enhanced therapeutic agent delivery to the Ras-mutant tumors.

The Ras gene is a frequently mutated gene that has been implicated in more than 20% of human cancers. The protein product of the Ras gene (Ras protein) is a small, 21 kDa guanosine triphosphatase (GTPase) involved in cell signaling and is active when bound to GTP and inactive when bound to GDP. Without wishing to be bound by any theory, it is believed that Ras proteins regulate cell growth, differentiation, and apoptosis by interacting with multiple effectors including mitogen-activated protein kinase (MAPF), phosphoinositide 3-kinase (PI3K) and signal transducer and activator of transcription (STAT) cascades.

Also without wishing to be bound by any theory, it is believed that, in at least some cases, Ras proteins acquire transforming potential when a point mutation in the RAS gene replaces an amino acid at position 12, 13, or 61. These mutations lead to forms of RAS with impaired GTPase activity, causing constitutive activation of RAS signalling pathway. Mutation of Ras can result in decreased GTPase activity, thereby prolonging the activity of activated Ras and promoting cell growth, cell division, and cancer cell survival.

Ras has at least three isoforms, K, N, and H, with K-Ras being the most frequently mutated. Consequently, K-Ras has been investigated as a potential target for cancer treatment. Mutations in K-RAS gene occur frequently in non-small cell lung cancer (NSCLC), with a higher frequency in in adenocarcinoma (20-30%) and lower frequency in squamous-cell carcinoma (about 7%).

In some cases, K-Ras transformation can lead to increased cytokines, metalloproteinases, and/or a proinflammatory tumor microenvironment with increased tumor angiogenesis. Without wishing to be bound by any theory, it is believed that increased leaky vasculature may promote enhanced permeability and retention of nanoparticles.

In some cases, K-Ras transformation can lead to increased macropinocytosis of proteins. Without wishing to be bound by any theory, it is believed that increased macropinocytosis of albumin exhibited by Ras-mutant cells may contribute to the cell's increased need for nutrients and may provide an additional supply of amino acids. This same mechanism could lead to increased nanoparticle uptake and/or increased uptake of released docetaxel that is protein-bound. To investigate the impact of macropinocytosis on nanoparticle uptake, a set of sequential studies can be conducted. For example: (1) Establish uptake conditions for disclosed nanoparticles in Ras wild-type and mutant cells and optimize the concentration of nanoparticles for visualization; (2) Establish incubation times for visualization; (3) Compare uptake in ˜5 wild-type and mutant cell lines; (4) If differences are observed, co-localize with markers of macropinocytosis; (5) Determine if ethylisopropylamiloride (EIPA), an inhibitor of macropinocytosis, inhibits uptake; and (6) Correlate uptake with activity of disclosed nanoparticles and dose response; for example, in vivo studies can be conducted using various models of wild-type and mutant Ras tumor xenografts and/or, e.g., experiments can be conducted to determine whether increased uptake observed in vitro can be visualized in vivo and whether uptake translates to enhanced anti-tumor activity.

In some cases, K-Ras transformation can lead to increased sensitivity to mitotic stress. Ras-mutant cells may be hypersensitive to various forms of mitotic stress, including taxanes. Without wishing to be bound by any theory, it is believed that if increased nanoparticle uptake provides higher levels of docetaxel to lung cancer cells, the lung cancer cells may be even further sensitized to docetaxel.

In some embodiments, K-Ras mutations could promote responsiveness to disclosed nanoparticles.

In some embodiments, therapeutic nanoparticles may be used to treat, alleviate, ameliorate, relieve, delay onset of, inhibit progression of, reduce severity of, and/or reduce incidence of one or more symptoms or features of a cancer having a Ras mutation (i.e., a Ras-mutant cancer). In some embodiments, therapeutic nanoparticles may be used to treat Ras-mutant solid tumors, e.g., Ras-mutant cancer and/or Ras-mutant cancer cells. For example, the Ras mutation may be a mutation in a Ras gene selected from the group consisting of H-Ras, K-Ras, and N-Ras. In some embodiments, a therapeutic agent formulated in a nanoparticle formulation may have increased efficacy for treating a Ras-mutant cancer as compared to free therapeutic agent, e.g., at least about 5% more effective, about 10% more effective, about 15% more effective, about 20% more effective, about 25% more effective, about 30% more effective, about 40% more effective, about 50% more effective, about 60% more effective, or about 75% more effective. In certain embodiments, a therapeutic agent formulated in a nanoparticle formulation may be between 5% and about 50% more effective for treating a Ras-mutant cancer as compared to free therapeutic agent.

In some embodiments, a nanoparticle composition may have increased efficacy for treating a Ras-mutant cancer as compared to a non-Ras-mutant cancer, e.g., at least about 5% more effective, about 10% more effective, about 15% more effective, about 20% more effective, about 25% more effective, about 30% more effective, about 40% more effective, about 50% more effective, about 60% more effective, or about 75% more effective. In certain embodiments, a nanoparticle composition may be between 5% and about 50% more effective for treating a Ras-mutant cancer as compared to a non-Ras-mutant cancer.

In certain embodiments, the response rate of patients having a Ras-mutant cancer may be greater than the response rate of patients having a non-Ras-mutant cancer when treated with a contemplated nanoparticle composition. For example, in some embodiments, the objective response rate of patients having a Ras-mutant cancer may be at least about 15%, in some embodiments at least about 18%, in some embodiments at least about 20%, in some embodiments at least about 22%, and in some embodiments at least about 25% when treated with a contemplated nanoparticle composition. In certain embodiments, the objective response rate of patients having a Ras-mutant cancer may be between about 15% and about 25%, in some embodiments between about 15% and about 20%, in some embodiments between about 18% and about 25%, and in some embodiments between about 20% and about 25% when treated with a contemplated nanoparticle composition.

As one of ordinary skill in the art would know, the response rate may be defined by the percentage of patients exhibiting complete response (CR) or partial response (PR) to a contemplated nanoparticle formulation. As used herein, CR refers to disappearance of all target lesions (e.g., tumors). Also as used herein, PR refers to a decrease of at least 30% in the sum of the longest diameter of target lesions as compared to the sum of the longest diameter of target lesions at baseline. Longest diameter of target lesions may be determined by any suitable technique. In certain embodiments, longest diameter of target lesions may be measured by computed tomography (i.e., CT).

In certain embodiments, the median duration of disease control (DC) in patients having a Ras-mutant cancer may be greater than the median duration of disease control in patients having a non-Ras-mutant cancer when treated with a contemplated nanoparticle composition. For example, in some embodiments, the median duration of disease control in patients having a Ras-mutant cancer may be at least about 5 months, in some embodiments at least about 5.5 months, in some embodiments at least about 6 months, in some embodiments at least about 6.5 months, and in some embodiments at least about 7 months when treated with a contemplated nanoparticle composition. In certain embodiments, the median duration of disease control in patients having a Ras-mutant cancer may be between about 5 months and about 9 months, in some embodiments between about 5 months and about 8 months, and in some embodiments between about 6 months and about 8 months when treated with a contemplated nanoparticle composition. In certain embodiments, the median duration of disease control in patients having a Ras-mutant cancer may be at least about 1 month, at least about 2 months, or at least about 3 months greater than the median duration of disease control in patients having a non-Ras-mutant cancer when treated with a contemplated nanoparticle composition. In certain embodiments, the median duration of disease control in patients having a Ras-mutant cancer may be between about 1 month and about 3 months greater or between about 2 months and about 3 months greater than the median duration of disease control in patients having a non-Ras-mutant cancer when treated with a contemplated nanoparticle composition.

In certain embodiments, the median survival of patients having a Ras-mutant cancer may be greater than the median survival of patients having a non-Ras-mutant cancer when treated with a contemplated nanoparticle composition. For example, in some embodiments, the median survival of patients having a Ras-mutant cancer may be at least about 6.5 months, in some embodiments at least about 7 months, in some embodiments at least about 7.5 months, in some embodiments at least about 8 months, in some embodiments at least about 9 months, and in some embodiments at least about 10 months when treated with a contemplated nanoparticle composition. In certain embodiments, the median survival of patients having a Ras-mutant cancer may be between about 6.5 months and about 11 months, in some embodiments between about 6.5 months and about 10 months, in some embodiments between about 7 months and about 10 months, in some embodiments between about 8 months and about 11 months, and in some embodiments between about 9 months and about 10 months when treated with a contemplated nanoparticle composition. In certain embodiments, the median survival of patients having a Ras-mutant cancer may be at least about 1 month, at least about 2 months, at least about 3 months, or at least about 4 months greater than the median survival of patients having a non-Ras-mutant cancer when treated with a contemplated nanoparticle composition. In certain embodiments, the median survival of patients having a Ras-mutant cancer may be between about 1 month and about 4 months greater, between about 2 months and about 4 months greater, or between about 3 months and about 4 months greater than the median survival of patients having a non-Ras-mutant cancer when treated with a contemplated nanoparticle composition.

In certain embodiments, therapeutic nanoparticles may be used to treat any cancer wherein PSMA is expressed on the surface of cancer cells or in the tumor neovasculature in a subject in need thereof, including the neovasculature of prostate or non-prostate solid tumors. Examples of the PSMA-related indication include, but are not limited to, prostate cancer, breast cancer, non-small cell lung cancer, colorectal carcinoma, and glioblastoma.

The term “cancer” includes pre-malignant as well as malignant cancers. Cancers that may be treated using the disclosed methods include, but are not limited to, blood cancer (e.g., myelodysplastic syndromes, acute myeloid leukemia, chronic myelogenous leukemia, chronic myelomonocytic leukemia, Philadelphia chromosome positive acute lymphoblastic leukemia, mantle cell lymphoma), oropharyngeal cancer, prostate cancer, cervical cancer, gastric cancer, anal cancer, colorectal cancer, gallbladder cancer, bile duct cancer, cancer of the bowel, skin cancer, e.g., melanomas or basal cell carcinomas, lung cancer (e.g., small-cell lung cancer or non-small cell lung cancer (e.g. adenocarcinoma, squamous cell carcinoma)), breast cancer, cancers of the head and neck, tonsillar cancer, bronchus cancer, pancreatic cancer, urinary bladder cancer, brain or central nervous system cancer, peripheral nervous system cancer, throat cancer, esophageal cancer, cancer of the oral cavity or pharynx, liver cancer (e.g., hepatocellular carcinoma), kidney cancer (e.g., renal cell carcinoma), testicular cancer, biliary tract cancer, small bowel or appendix cancer, gastrointestinal stromal tumor, salivary gland cancer, thyroid gland cancer (e.g., follicular thyroid cancer and undifferentiated thyroid cancer), adrenal gland cancer, osteosarcoma, chondrosarcoma, cancer of hematological tissues, and the like. “Cancer cells” can be in the form of a tumor (i.e., a solid tumor), exist alone within a subject (e.g., leukemia cells), or be cell lines derived from a cancer.

Cancer can be associated with a variety of physical symptoms. Symptoms of cancer generally depend on the type and location of the tumor. For example, lung cancer can cause coughing, shortness of breath, and chest pain, while colon cancer often causes diarrhea, constipation, and blood in the stool. However, to give but a few examples, the following symptoms are often generally associated with many cancers: fever, chills, night sweats, cough, dyspnea, weight loss, loss of appetite, anorexia, nausea, vomiting, diarrhea, anemia, jaundice, hepatomegaly, hemoptysis, fatigue, malaise, cognitive dysfunction, depression, hormonal disturbances, neutropenia, pain, non-healing sores, enlarged lymph nodes, peripheral neuropathy, and sexual dysfunction.

Also provided herein are methods of administering to a patient nanoparticles disclosed herein that include a therapeutic agent, wherein, upon administration to a patient, such nanoparticles substantially reduce the volume of distribution and/or substantially reduces free Cmax, as compared to administration of the agent alone (i.e. not as a disclosed nanoparticle).

Disclosed methods may include administration of a disclosed nanoparticle composition, wherein the composition is administered over a period of three weeks, a month, or two months or more. For example, disclosed herein are methods of treating cancers that include administering a disclosed nanoparticle composition over a period of at least two weeks, three weeks, one month or administered over a period of about 2 weeks to about 6 months or more, wherein the interval between each administration is no more than about once a day, once a week, once every two weeks, once every three weeks, or once every month, and wherein the dose of the active agent (e.g. docetaxel) at each administration is about 30 mg/m² to about 75 mg/m², or about 50 mg/m² to about 75 mg/m², or about 60 mg/m² to about 70 mg/m² or about 55 mg/m² or about 60 mg/m².

Also provided herein is a method of treating a cancer (e.g. refractory cancer) in a patient in need thereof, comprising administering to the patient a therapeutically effective amount of a disclosed nanoparticle composition. Such a refractory cancer may be any cancer having a mutation in a Ras gene (e.g., in the H-Ras, K-Ras, and/or N-Ras genes). A refractory cancer may be a cancer that has been treated previously in a patient with one or more chemotherapeutics and/or radiation, but that is not responsive to those first line therapies. Non-limiting examples of the refractory cancers include adenocarcinomas selected from lung, colon, and pancreatic cancer; follicular thyroid cancer; undifferentiated thyroid cancer; myelodysplastic syndromes; and acute myeloid leukemia.

Contemplated herein are method of treating cancers, e.g. refractory cancers, in a patient comprising administering a) an effective amount of a disclosed nanoparticle composition comprising a therapeutic agent (e.g. docetaxel) and optionally b) an effective amount of at least one other chemotherapeutic agent. In some embodiments, the other chemotherapeutic agent may be cisplatin, capecitabine, oxaliplatin, gemcitabine, 5-fluorouracil (5FU), mitomycin, gemcitabine, or a combination of other chemotherapeutic agents. In such combination therapies, the composition comprising nanoparticles and the other chemotherapeutic agent can be administered simultaneously, either in the same composition or in separate compositions, administered sequentially, i.e., the nanoparticle composition can be administered either prior to or after the administration of the other chemotherapeutic agent. In some embodiments, the administration of the nanoparticle composition and the chemotherapeutic agent can be concurrent, i.e., the administration period of the nanoparticle composition and that of the chemotherapeutic agent overlap with each other. In some embodiments, the administration of the nanoparticle composition and the chemotherapeutic agent are non-concurrent. For example, in some embodiments, the administration of the nanoparticle composition is terminated before the chemotherapeutic agent is administered. In some embodiments, the administration of the other chemotherapeutic agent is terminated before the nanoparticle composition is administered. In a method of treating refractory cancer, the patient may not have been responsive to the other agents.

Also contemplated are methods of treating Ras-mutant cancers comprising administering an effective amount of a disclosed nanoparticle composition and administering a therapy that activates the nanoparticle composition. For example, a patient may be treated with a thermal anti-cancer therapy using, e.g., infrared, radio frequency, or a magnetic field, which heat an administered nanoparticle composition. For instance, radio frequency may be used to heat, e.g., gold or iron oxide nanoparticles and a magnetic field may be used to heat, e.g., iron oxide nanoparticles.

Methods of treating cancer that are also contemplated include a) a first therapy comprising administering to a patient a disclosed nanoparticle composition, and b) a second therapy comprising radiation therapy, surgery, or combinations thereof.

In certain embodiments, a contemplated method may comprise administering a contemplated nanoparticle composition to a patient having a Ras-mutant cancer using a treatment cycle. In certain embodiments, a contemplated nanoparticle composition may be administered once per treatment cycle. In some embodiments, a treatment cycle may be 1-3 days, in some embodiments 3-5 days, in some embodiments 3-7 days, in some embodiments 3-10 days, in some embodiments 5-15 days, in some embodiments 10-20 days, in some embodiments 15-25 days, in some embodiments 20-30 days, or in some embodiments 1-30 days in length. For example, in one embodiment, a contemplated treatment cycle may be 21 days long.

In certain embodiments, a treatment cycle may be repeated. For example, the number of treatment cycles may be at least 2, in some embodiments at least 3, in some embodiments at least 4, in some embodiments at least 5, in some embodiments at least 6, in some embodiments at least 7, in some embodiments at least 8, in some embodiments at least 9, or in some embodiments at least 10. In certain embodiments, the number of treatment cycles may be between 1 and 15, in some embodiments between 1 and 10, in some embodiments between 5 and 15, in some embodiments between 10 and 15, in some embodiments between 1 and 5, in some embodiments between 2 and 10, in some embodiments between 2 and 8, or in some embodiments between 2 and 6. In certain embodiments, the number of treatment cycles may be 4.

In certain embodiments, a contemplated method may include determining a patient's Ras mutation status. For example, in some embodiments, a patient may be tested for the presence or absence of a Ras mutation (e.g., a K-Ras mutation), and upon diagnosis as having a Ras mutation, be administered a contemplated nanoparticle composition. For instance, a candidate patient may be tested for a Ras mutation and then administered a nanoparticle composition comprising therapeutic nanoparticles if the patient is determined to have a Ras mutation. In some embodiments, a sample may be taken from the patient and subjected to a diagnostic assay to determine the presence or absence of a Ras mutation (e.g., a K-Ras mutation). For example, in some instances, the sample may be a blood, serum, plasma, saliva, urine, semen, or stool sample. In other embodiments, the sample may be a tissue sample, such as, for example, a tumor biopsy.

Tests for Ras mutation status are known in the art and include, for example, polymerase chain reaction (PCR) and nucleic acid (e.g., DNA) sequencing to detect nucleotide mutations that correspond to mutations in the amino acid sequence of a Ras protein. Non-limiting examples of K-Ras nucleotide mutations include c.35G>A, c.34G>T, c.34G>A, c.34G>C, c.35G>C, c.35G>T, c.38G>A, c.183A>C, c.183A>T, c.182A>T, c.182A>G, c.351A>C, c.351A>T, c.350A>G, c.349A>G, c.436G>A, c.436G>C, and c.437C>T. Non-limiting examples of K-Ras protein mutations include G12D, G12C, G12S, G12R, G12A, G12V, G13D, Q61H, Q61L, Q61R, K117N, K117R, K117E, A146T, A146P, and A146V.

### Nanoparticles

Nanoparticles disclosed herein may be any particles capable of exhibiting an anti-cancer effect. Non-limiting examples of nanoparticles include core-shell nanoparticles, liposomes, micelles, nanocrystals, and solid particles. Nanoparticles may be, but are not limited to, polymeric nanoparticles, degradable nanoparticles, non-degradable nanoparticles, cyclodextrin-based nanoparticles, ceramic nanoparticles (e.g., oxides, such as silica and iron oxide, and nitrides), inorganic nanoparticles, metal nanoparticles (e.g., gold), lipid-based nanoparticles (e.g., liposomes), magnetic nanoparticles, quantum dots, and dendrimers. For example, in some embodiments, a contemplated nanoparticle may comprise a polymer, an inorganic compound, a metal or metal alloy, a lipid, a paramagnetic material, a ceramic, a semi-conductor material, or a dendrimer. In some embodiments, the nanoparticles may comprise a therapeutic agent. For example, the therapeutic agent may be incorporated in a coating surrounding the nanoparticle or the therapeutic agent may be encapsulated by, associated with, dispersed within, and/or covalently bonded to the nanoparticle. In some embodiments, a nanoparticle may be formed from a therapeutic anti-cancer agent, e.g., the nanoparticle may be a pure drug nanoparticle, such as an amorphous or crystalline drug particle.

In some embodiments, nanoparticles disclosed herein may include one, two, three or more biocompatible and/or biodegradable polymers. For example, a contemplated nanoparticle may include about 35 to about 99.75 weight percent, in some embodiments about 50 to about 99.75 weight percent, in some embodiments about 50 to about 99.5 weight percent, in some embodiments about 50 to about 99 weight percent, in some embodiments about 50 to about 98 weight percent, in some embodiments about 50 to about 97 weight percent, in some embodiments about 50 to about 96 weight percent, in some embodiments about 50 to about 95 weight percent, in some embodiments about 50 to about 94 weight percent, in some embodiments about 50 to about 93 weight percent, in some embodiments about 50 to about 92 weight percent, in some embodiments about 50 to about 91 weight percent, in some embodiments about 50 to about 90 weight percent, in some embodiments about 50 to about 85 weight percent, in some embodiments about 60 to about 85 weight percent, in some embodiments about 65 to about 85 weight percent, and in some embodiments about 50 to about 80 weight percent of one or more block copolymers that include a biodegradable polymer and poly(ethylene glycol) (PEG), and about 0 to about 50 weight percent of a biodegradable homopolymer.

In some embodiments, disclosed nanoparticles may include about 0.2 to about 35 weight percent, about 0.2 to about 20 weight percent, about 0.2 to about 10 weight percent, about 0.2 to about 5 weight percent, about 0.5 to about 5 weight percent, about 0.75 to about 5 weight percent, about 1 to about 5 weight percent, about 2 to about 5 weight percent, about 3 to about 5 weight percent, about 1 to about 20 weight percent, about 2 to about 20 weight percent, about 4 to about 20 weight percent, about 5 to about 20 weight percent, about 10 to about 20 weight percent, about 1 to about 15 weight percent, about 2 to about 15 weight percent, about 3 to about 15 weight percent, about 4 to about 15 weight percent, about 5 to about 15 weight percent, about 1 to about 10 weight percent, about 2 to about 10 weight percent, about 3 to about 10 weight percent, about 4 to about 10 weight percent, about 5 to about 10 weight percent, about 10 to about 30 weight percent, or about 15 to about 25 weight percent of a therapeutic agent.

In some embodiments, disclosed nanoparticles substantially immediately release (e.g., over about 1 minute to about 30 minutes, about 1 minute to about 25 minutes, about 5 minutes to about 30 minutes, about 5 minutes to about 1 hour, about 1 hour, or about 24 hours) less than about 2%, less than about 5%, less than about 10%, less than about 15%, less than about 20%, less than about 25%, less than about 30%, or less than 40% of the therapeutic agent, for example when placed in a phosphate buffer solution at room temperature (e.g., 25° C.) and/or at 37° C. In certain embodiments, nanoparticles comprising a therapeutic agent may release the therapeutic agent when placed in an aqueous solution (e.g., a phosphate buffer solution), e.g., at 25° C. and/or at 37° C., at a rate substantially corresponding to about 0.01 to about 50%, in some embodiments about 0.01 to about 25%, in some embodiments about 0.01 to about 15%, in some embodiments about 0.01 to about 10%, in some embodiments about 1 to about 40%, in some embodiments about 5 to about 40%, and in some embodiments about 10 to about 40% of the therapeutic agent released over about 1 hour. In some embodiments, nanoparticles comprising a therapeutic agent may release the therapeutic agent when placed in an aqueous solution (e.g., a phosphate buffer solution), e.g., at 25° C. and/or at 37° C., at a rate substantially corresponding to about 10 to about 70%, in some embodiments about 10 to about 45%, in some embodiments about 10 to about 35%, or in some embodiments about 10 to about 25%, of the therapeutic agent released over about 4 hours.

In some embodiments, disclosed nanoparticles may substantially retain the therapeutic agent, e.g., for at least about 1 minute, at least about 1 hour, or more, when placed in a phosphate buffer solution at 37° C.

In some embodiments, a contemplated nanoparticle may comprise a cyclodextrin. A suitable cyclodextrin may include α-cyclodextrin, β-cyclodextrin, γ-cyclodextrin, or mixtures thereof. Exemplary cyclodextrins contemplated for use in the nanoparticles disclosed herein include hydroxypropyl-β-cyclodextrin (HPbCD), hydroxyethyl-β-cyclodextrin, sulfobutylether-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-β-cyclodextrin, glucosyl-β-cyclodextrin, and maltosyl-β-cyclodextrin. In some embodiments, the cyclodextrin may be covalently attached to polymer. For example, in some embodiments, the cyclodextrin may be covalently attached to chitosan.

In one embodiment, disclosed therapeutic nanoparticles may include a targeting ligand, e.g., a low-molecular weight ligand. In certain embodiments, the low-molecular weight ligand is conjugated to a polymer, and the nanoparticle comprises a certain ratio of ligand-conjugated polymer (e.g., PLA-PEG-Ligand) to non-functionalized polymer (e.g., PLA-PEG or PLGA-PEG). The nanoparticle can have an optimized ratio of these two polymers such that an effective amount of ligand is associated with the nanoparticle for treatment of a disease or disorder, such as cancer. For example, an increased ligand density may increase target binding (cell binding/target uptake), making the nanoparticle “target specific.” Alternatively, a certain concentration of non-functionalized polymer (e.g., non-functionalized PLGA-PEG copolymer) in the nanoparticle can control inflammation and/or immunogenicity (i.e., the ability to provoke an immune response), and allow the nanoparticle to have a circulation half-life that is adequate for the treatment of a disease or disorder. Furthermore, the non-functionalized polymer may, in some embodiments, lower the rate of clearance from the circulatory system via the reticuloendothelial system (RES). Thus, the non-functionalized polymer may provide the nanoparticle with characteristics that may allow the particle to travel through the body upon administration. In some embodiments, a non-functionalized polymer may balance an otherwise high concentration of ligands, which can otherwise accelerate clearance by the subject, resulting in less delivery to the target cells.

In some embodiments, nanoparticles disclosed herein may include functionalized polymers conjugated to a ligand that constitute approximately 0.1-50, e.g., 0.1-30, e.g., 0.1-20, e.g., 0.1-10 mole percent of the entire polymer composition of the nanoparticle (i.e., functionalized+non-functionalized polymer). Also disclosed herein, in another embodiment, are nanoparticles that include a polymer conjugated (e.g., covalently with (i.e., through a linker (e.g., an alkylene linker)) or a bond) with one or more low-molecular weight ligands, wherein the weight percent low-molecular weight ligand with respect to total polymer is between about 0.001 and 5, e.g., between about 0.001 and 2, e.g., between about 0.001 and 1.

In some embodiments, disclosed nanoparticles may be able to bind efficiently to or otherwise associate with a biological entity, for example, a particular membrane component or cell surface receptor. Targeting of a therapeutic agent (e.g., to a particular tissue or cell type, to a specific diseased tissue but not to normal tissue, etc.) is desirable for the treatment of tissue specific diseases such as solid tumor cancers (e.g., prostate cancer). For example, in contrast to systemic delivery of a cytotoxic anti-cancer agent, the nanoparticles disclosed herein may substantially prevent the agent from killing healthy cells. Additionally, disclosed nanoparticles may allow for the administration of a lower dose of the agent (as compared to an effective amount of agent administered without disclosed nanoparticles or formulations) which may reduce the undesirable side effects commonly associated with traditional chemotherapy.

In general, a “nanoparticle” refers to any particle having a diameter of less than 1000 nm, e.g., about 10 nm to about 200 nm. Disclosed therapeutic nanoparticles may include nanoparticles having a diameter of about 60 to about 120 nm, or about 70 to about 120 nm, or about 80 to about 120 nm, or about 90 to about 120 nm, or about 100 to about 120 nm, or about 60 to about 130 nm, or about 70 to about 130 nm, or about 80 to about 130 nm, or about 90 to about 130 nm, or about 100 to about 130 nm, or about 110 to about 130 nm, or about 60 to about 140 nm, or about 70 to about 140 nm, or about 80 to about 140 nm, or about 90 to about 140 nm, or about 100 to about 140 nm, or about 110 to about 140 nm, or about 60 to about 150 nm, or about 70 to about 150 nm, or about 80 to about 150 nm, or about 90 to about 150 nm, or about 100 to about 150 nm, or about 110 to about 150 nm, or about 120 to about 150 nm.

### Polymers

In some embodiments, the nanoparticles may comprise a matrix of polymers and a therapeutic agent. In some embodiments, a therapeutic agent and/or targeting moiety (i.e., a low-molecular weight ligand) can be associated with at least part of the polymeric matrix. For example, in some embodiments, a targeting moiety (e.g., ligand) can be covalently associated with the surface of a polymeric matrix. In some embodiments, covalent association is mediated by a linker. The therapeutic agent can be associated with the surface of, encapsulated within, surrounded by, and/or dispersed throughout the polymeric matrix.

A wide variety of polymers and methods for forming particles therefrom are known in the art of drug delivery. In some embodiments, the disclosure is directed toward nanoparticles with at least two macromolecules, wherein the first macromolecule comprises a first polymer bound to a low-molecular weight ligand (e.g., targeting moiety); and the second macromolecule comprising a second polymer that is not bound to a targeting moiety. The nanoparticle can optionally include one or more additional, unfunctionalized, polymers.

Any suitable polymer can be used in the disclosed nanoparticles. Polymers can be natural or unnatural (synthetic) polymers. Polymers can be homopolymers or copolymers comprising two or more monomers. In terms of sequence, copolymers can be random, block, or comprise a combination of random and block sequences. Typically, polymers are organic polymers.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments include polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and ether bonds (e.g., R—O—R′ bonds). In some embodiments, a biodegradable polymer, such as a hydrolyzable polymer, containing carboxylic acid groups, may be conjugated with poly(ethylene glycol) repeat units to form a poly(ester-ether). A polymer (e.g., copolymer, e.g., block copolymer) containing poly(ethylene glycol) repeat units can also be referred to as a “PEGylated” polymer.

For instance, a contemplated polymer may be one that hydrolyzes spontaneously upon exposure to water (e.g., within a subject), or the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof). In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g., DNA, RNA, or derivatives thereof). Amine-containing polymers such as poly(lysine), polyethylene imine (PEI), and poly(amidoamine) dendrimers are contemplated for use, in some embodiments, in a disclosed particle.

In some embodiments, polymers can be degradable polyesters bearing cationic side chains. Examples of these polyesters include poly(L-lactide-co-L-lysine), poly(serine ester), poly(4-hydroxy-L-proline ester).

It is contemplated that PEG may be terminated and include an end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

In one embodiment, the molecular weight (or e.g., the ratio of molecular weights of, e.g., different blocks of a copolymer) of the polymers can be optimized for effective treatment as disclosed herein. For example, the molecular weight of a polymer may influence particle degradation rate (such as when the molecular weight of a biodegradable polymer can be adjusted), solubility, water uptake, and drug release kinetics. For example, the molecular weight of the polymer (or e.g., the ratio of molecular weights of, e.g., different blocks of a copolymer) can be adjusted such that the particle biodegrades in the subject being treated within a reasonable period of time (ranging from a few hours to 1-2 weeks, 3-4 weeks, 5-6 weeks, 7-8 weeks, etc.).

A disclosed particle can for example comprise a diblock copolymer of PEG and PL(G)A, wherein for example, the PEG portion may have a number average molecular weight of about 1,000-20,000, e.g., about 2,000-20,000, e.g., about 2 to about 10,000, and the PL(G)A portion may have a number average molecular weight of about 5,000 to about 20,000, or about 5,000-100,000, e.g., about 20,000-70,000, e.g., about 15,000-50,000.

For example, disclosed here is an exemplary therapeutic nanoparticle that includes about 10 to about 99 weight percent poly(lactic) acid-poly(ethylene)glycol copolymer or poly(lactic)-co-poly (glycolic) acid-poly(ethylene)glycol copolymer, or about 20 to about 80 weight percent, about 40 to about 80 weight percent, or about 30 to about 50 weight percent, or about 70 to about 90 weight percent poly(lactic) acid-poly(ethylene)glycol copolymer or poly(lactic)-co-poly (glycolic) acid-poly(ethylene)glycol copolymer. Exemplary poly(lactic) acid-poly(ethylene)glycol copolymers can include a number average molecular weight of about 15 to about 20 kDa, or about 10 to about 25 kDa of poly(lactic) acid and a number average molecular weight of about 4 to about 6, or about 2 kDa to about 10 kDa of poly(ethylene)glycol.

In some embodiments, the poly(lactic) acid-poly(ethylene)glycol copolymer may have a poly(lactic) acid number average molecular weight fraction of about 0.6 to about 0.95, in some embodiments between about 0.7 to about 0.9, in some embodiments between about 0.6 to about 0.8, in some embodiments between about 0.7 to about 0.8, in some embodiments between about 0.75 to about 0.85, in some embodiments between about 0.8 to about 0.9, and in some embodiments between about 0.85 to about 0.95. It should be understood that the poly(lactic) acid number average molecular weight fraction may be calculated by dividing the number average molecular weight of the poly(lactic) acid component of the copolymer by the sum of the number average molecular weight of the poly(lactic) acid component and the number average molecular weight of the poly(ethylene)glycol component.

Disclosed nanoparticles may optionally include about 1 to about 50 weight percent poly(lactic) acid or poly(lactic) acid-co-poly (glycolic) acid (which does not include PEG), or may optionally include about 1 to about 50 weight percent, or about 10 to about 50 weight percent or about 30 to about 50 weight percent poly(lactic) acid or poly(lactic) acid-co-poly (glycolic) acid. For example, poly(lactic) or poly(lactic)-co-poly(glycolic) acid may have a number average molecule weight of about 5 to about 15 kDa, or about 5 to about 12 kDa. Exemplary PLA may have a number average molecular weight of about 5 to about 10 kDa. Exemplary PLGA may have a number average molecular weight of about 8 to about 12 kDa.

A therapeutic nanoparticle may, in some embodiments, contain about 10 to about 30 weight percent, in some embodiments about 10 to about 25 weight percent, in some embodiments about 10 to about 20 weight percent, in some embodiments about 10 to about 15 weight percent, in some embodiments about 15 to about 20 weight percent, in some embodiments about 15 to about 25 weight percent, in some embodiments about 20 to about 25 weight percent, in some embodiments about 20 to about 30 weight percent, or in some embodiments about 25 to about 30 weight percent of poly(ethylene)glycol, where the poly(ethylene)glycol may be present as a poly(lactic) acid-poly(ethylene)glycol copolymer, poly(lactic)-co-poly (glycolic) acid-poly(ethylene)glycol copolymer, or poly(ethylene)glycol homopolymer. In certain embodiments, the polymers of the nanoparticles can be conjugated to a lipid. The polymer can be, for example, a lipid-terminated PEG.

### Targeting Moieties

Provided herein, in some embodiments, are nanoparticles that may include an optional targeting moiety, i.e., a moiety able to bind to or otherwise associate with a biological entity, for example, a membrane component, a cell surface receptor, an antigen, or the like. A targeting moiety present on the surface of the particle may allow the particle to become localized at a particular targeting site, for instance, a tumor, a disease site, a tissue, an organ, a type of cell, etc. As such, the nanoparticle may then be “target specific.” The drug or other payload may then, in some cases, be released from the particle and allowed to interact locally with the particular targeting site.

In one embodiment, a disclosed nanoparticle includes a targeting moiety that is a low-molecular weight ligand. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

For example, a targeting portion may cause the particles to become localized to a tumor (e.g., a solid tumor), a disease site, a tissue, an organ, a type of cell, etc. within the body of a subject, depending on the targeting moiety used. For example, a low-molecular weight ligand may become localized to a solid tumor, e.g., breast or prostate tumors or cancer cells. The subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

Contemplated targeting moieties may include small molecules. In certain embodiments, the term “small molecule” refers to organic compounds, whether naturally-occurring or artificially created (e.g., via chemical synthesis) that have relatively low molecular weight and that are not proteins, polypeptides, or nucleic acids. Small molecules typically have multiple carbon-carbon bonds. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol, for example about 100 g/mol to about 600 g/mol, or about 200 g/mol to about 500 g/mol.

In some embodiments, the low-molecular weight ligand is of the Formulae I, II, III or IV:

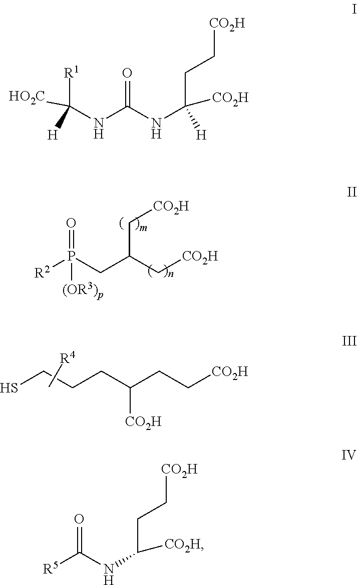

and enantiomers, stereoisomers, rotamers, tautomers, diastereomers, or racemates thereof;

wherein m and n are each, independently, 0, 1, 2 or 3; p is 0 or 1;

R¹, R², R⁴, and R⁵ are each, independently, selected from the group consisting of substituted or unsubstituted alkyl (e.g., C₁₋₁₀-alkyl, C₁₋₆-alkyl, or C₁₋₄-alkyl), substituted or unsubstituted aryl (e.g., phenyl or pyridinyl), and any combination thereof; and R³ is H or C₁₋₆-alkyl (e.g., CH₃).

For compounds of Formulae I, II, III and IV, R¹, R², R⁴ or R⁵ comprise points of attachment to the nanoparticle, e.g., a point of attachment to a polymer that forms part of a disclosed nanoparticle, e.g., PEG. The point of attachment may be formed by a covalent bond, ionic bond, hydrogen bond, a bond formed by adsorption including chemical adsorption and physical adsorption, a bond formed from van der Waals bonds, or dispersion forces. For example, if R¹, R², R⁴, or R⁵ are defined as an aniline or C₁₋₆-alkyl-NH₂ group, any hydrogen (e.g., an amino hydrogen) of these functional groups could be removed such that the low-molecular weight ligand is covalently bound to the polymeric matrix (e.g., the PEG-block of the polymeric matrix) of the nanoparticle. As used herein, the term “covalent bond” refers to a bond between two atoms formed by sharing at least one pair of electrons.

In particular embodiments of the Formulae I, II, III or IV, R¹, R², R⁴, and R⁵ are each, independently, C₁₋₆-alkyl or phenyl, or any combination of C₁₋₆-alkyl or phenyl, which are independently substituted one or more times with OH, SH, NH₂, or CO₂H, and wherein the alkyl group may be interrupted by N(H), S, or O. In another embodiment, R¹, R², R⁴, and R⁵ are each, independently, CH₂-Ph, (CH₂)₂—SH, CH₂—SH, (CH₂)₂C(H)(NH₂)CO₂H, CH₂C(H)(NH₂)CO₂H, CH(NH₂)CH₂CO₂H, (CH₂)₂C(H)(SH)CO₂H, CH₂—N(H)-Ph, O—CH₂-Ph, or O—(CH₂)₂-Ph, wherein each Ph may be independently substituted one or more times with OH, NH₂, CO₂H, or SH. For these formulae, the NH₂, OH or SH groups serve as the point of covalent attachment to the nanoparticle (e.g., —N(H)—PEG, —O-PEG, or —S-PEG).

Exemplary ligands include:

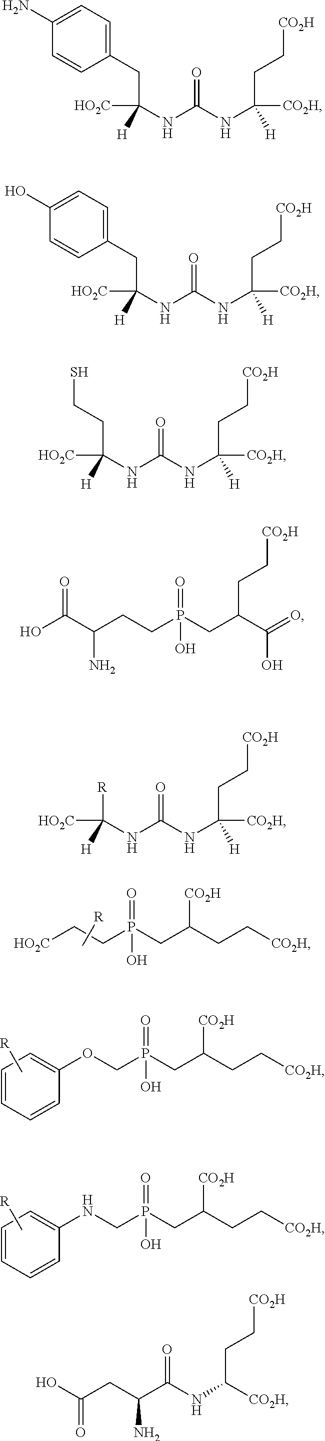

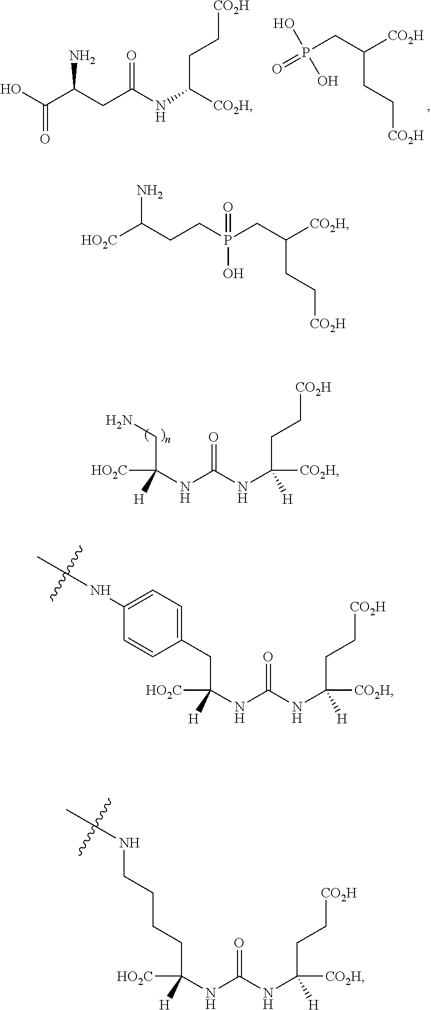

and enantiomers, stereoisomers, rotamers, tautomers, diastereomers, or racemates thereof, wherein the NH₂, OH, or SH groups serve as the point of covalent attachment to the nanoparticle (e.g., —N(H)—PEG, —O-PEG, or —S-PEG) or

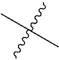

indicates the point of attachment to the nanoparticle, wherein n is 1, 2, 3, 4, 5, or 6, and wherein R is independently selected from the group consisting of NH₂, SH, OH, CO₂H, C₁₋₆-alkyl that is substituted with NH₂, SH, OH, or CO₂H, and phenyl that is substituted with NH₂, SH, OH, or CO₂H, and wherein R serves as the point of covalent attachment to the nanoparticle (e.g., —N(H)—PEG, —S-PEG, —O-PEG, or CO₂—PEG). These compounds may be further substituted with NH₂, SH, OH, CO₂H, C₁₋₆-alkyl that is substituted with NH₂, SH, OH, or CO₂H, or phenyl that is substituted with NH₂, SH, OH or CO₂H, wherein these functional groups can also serve as the point of covalent attachment to the nanoparticle.

In some embodiments, small molecule targeting moieties that may be used to target cells associated with solid tumors such as prostate or breast cancer tumors include PSMA peptidase inhibitors such as 2-PMPA, GPI5232, VA-033, phenylalkylphosphonamidates and/or analogs and derivatives thereof. In some embodiments, small molecule targeting moieties that may be used to target cells associated with prostate cancer tumors include thiol and indole thiol derivatives, such as 2-MPPA and 3-(2-mercaptoethyl)-1H-indole-2-carboxylic acid derivatives. In some embodiments, small molecule targeting moieties that may be used to target cells associated with prostate cancer tumors include hydroxamate derivatives. In some embodiments, small molecule targeting moieties that may be used to target cells associated with prostate cancer tumors include PBDA- and urea-based inhibitors, such as ZJ 43, ZJ 11, ZJ 17, ZJ 38 and/or and analogs and derivatives thereof, androgen receptor targeting agents (ARTAs), polyamines, such as putrescine, spermine, and spermidine, inhibitors of the enzyme glutamate carboxylase II (GCPII), also known as NAAG Peptidase or NAALADase.

In another embodiment, the targeting moiety can be a ligand that targets Her2, EGFR, folate receptor or toll receptors. In another embodiment, the targeting moiety is folate, folic acid, or an EGFR binding molecule.

For example, contemplated the targeting moieties may include a nucleic acid, polypeptide, glycoprotein, carbohydrate, or lipid. For example, a targeting moiety can be a nucleic acid targeting moiety (e.g. an aptamer, e.g., the A10 aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments. Characteristic portions of antibodies, single chain targeting moieties can be identified, e.g., using procedures such as phage display.

Targeting moieties may be a targeting peptide or targeting peptidomimetic that has a length of up to about 50 residues. For example, a targeting moiety may include the amino acid sequence AKERC, CREKA, ARYLQKLN, or AXYLZZLN, wherein X and Z are variable amino acids, or conservative variants or peptidomimetics thereof. In particular embodiments, the targeting moiety is a peptide that includes the amino acid sequence AKERC, CREKA, ARYLQKLN, or AXYLZZLN, wherein X and Z are variable amino acids, and has a length of less than 20, 50 or 100 residues. The CREKA (Cys Arg Glu Lys Ala) peptide or a peptidomimetic thereof or the octapeptide AXYLZZLN are also contemplated as targeting moieties, as well as peptides, or conservative variants or peptidomimetics thereof, that bind or form a complex with collagen IV, or that target tissue basement membrane (e.g., the basement membrane of a blood vessel). Exemplary targeting moieties include peptides that target ICAM (intercellular adhesion molecule, e.g., ICAM-1).

Targeting moieties disclosed herein can be, in some embodiments, conjugated to a disclosed polymer or copolymer (e.g., PLA-PEG), and such a polymer conjugate may form part of a disclosed nanoparticle.

In some embodiments, a therapeutic nanoparticle may include a polymer-drug conjugate. For example, a drug may be conjugated to a disclosed polymer or copolymer (e.g., PLA-PEG), and such a polymer-drug conjugate may form part of a disclosed nanoparticle. For example, a disclosed therapeutic nanoparticle may optionally include about 0.2 to about 30 weight percent of a PLA-PEG or PLGA-PEG, wherein the PEG is functionalized with a drug (e.g., PLA-PEG-Drug).

A disclosed polymeric conjugate (e.g., a polymer-ligand conjugate) may be formed using any suitable conjugation technique. For instance, two compounds such as a targeting moiety or drug and a biocompatible polymer (e.g., a biocompatible polymer and a poly(ethylene glycol)) may be conjugated together using techniques such as EDC-NHS chemistry (1-ethyl-3-(3-dimethylaminopropyl) carbodiimide hydrochloride and N-hydroxysuccinimide) or a reaction involving a maleimide or a carboxylic acid, which can be conjugated to one end of a thiol, an amine, or a similarly functionalized polyether. The conjugation of a targeting moiety or drug and a polymer to form a polymer-targeting moiety conjugate or a polymer-drug conjugate can be performed in an organic solvent, such as, but not limited to, dichloromethane, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting a polymer that comprises a carboxylic acid functional group (e.g., a poly(ester-ether) compound) with a polymer or other moiety (such as a targeting moiety or drug) comprising an amine. For instance, a targeting moiety, such as a low-molecular weight ligand, or a drug, such as dasatinib, may be reacted with an amine to form an amine-containing moiety, which can then be conjugated to the carboxylic acid of the polymer. Such a reaction may occur as a single-step reaction, i.e., the conjugation is performed without using intermediates such as N-hydroxysuccinimide or a maleimide. In some embodiments, a drug may be reacted with an amine-containing linker to form an amine-containing drug, which can then be conjugated to the carboxylic acid of the polymer as described above. The conjugation reaction between the amine-containing moiety and the carboxylic acid-terminated polymer (such as a poly(ester-ether) compound) may be achieved, in one set of embodiments, by adding the amine-containing moiety, solubilized in an organic solvent such as (but not limited to) dichloromethane, acetonitrile, chloroform, tetrahydrofuran, acetone, formamide, dimethylformamide, pyridines, dioxane, or dimethylsulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously, in some cases. Unconjugated reactants may be washed away after such reactions, and the polymer may be precipitated in solvents such as, for instance, ethyl ether, hexane, methanol, or ethanol. In certain embodiments, a conjugate may be formed between an alcohol-containing moiety and carboxylic acid functional group of a polymer, which can be achieved similarly as described above for conjugates of amines and carboxylic acids.

### Preparation of Nanoparticles

Another aspect of this disclosure is directed to systems and methods of making disclosed nanoparticles. In some embodiments, using two or more different polymers (e.g., copolymers, e.g., block copolymers) in different ratios and producing particles from the polymers (e.g., copolymers, e.g., block copolymers), properties of the particles be controlled. For example, one polymer (e.g., copolymer, e.g., block copolymer) may include a low-molecular weight ligand, while another polymer (e.g., copolymer, e.g., block copolymer) may be chosen for its biocompatibility and/or its ability to control immunogenicity of the resultant particle.

In some embodiments, a solvent used in a nanoparticle preparation process (e.g., a nanoprecipitation process or a nanoemulsion process as discussed below) may include a hydrophobic acid or a hydrophobic base, which may confer advantageous properties to the nanoparticles prepared using the process. For example, in some cases, the hydrophobic acid or hydrophobic base may improve drug loading of disclosed nanoparticles. Furthermore, in some instances, the controlled release properties of disclosed nanoparticles may be improved by the use of the hydrophobic acid or hydrophobic base. In some cases, the hydrophobic acid or hydrophobic base may be included in, for example, an organic solution or an aqueous solution used in the process. In one embodiment, the drug is combined with an organic solution and the hydrophobic acid or hydrophobic base and optionally one or more polymers. The hydrophobic acid or hydrophobic base concentration in a solution used to dissolve the drug may be, for example, between about 1 weight percent and about 30 weight percent, etc.

In one set of embodiments, the particles are formed by providing a solution comprising one or more polymers, and contacting the solution with a polymer nonsolvent to produce the particle. The solution may be miscible or immiscible with the polymer nonsolvent. For example, a water-miscible liquid such as acetonitrile may contain the polymers, and particles are formed as the acetonitrile is contacted with water, a polymer nonsolvent, e.g., by pouring the acetonitrile into the water at a controlled rate. The polymer contained within the solution, upon contact with the polymer nonsolvent, may then precipitate to form particles such as nanoparticles. Two liquids are said to be “immiscible” or not miscible, with each other when one is not soluble in the other to a level of at least 10% by weight at ambient temperature and pressure. Typically, an organic solution (e.g., dichloromethane, acetonitrile, chloroform, tetrahydrofuran, acetone, formamide, dimethylformamide, pyridines, dioxane, dimethylsulfoxide, etc.) and an aqueous liquid (e.g., water, or water containing dissolved salts or other species, cell or biological media, ethanol, etc.) are immiscible with respect to each other. For example, the first solution may be poured into the second solution (at a suitable rate or speed). In some cases, particles such as nanoparticles may be formed as the first solution contacts the immiscible second liquid, e.g., precipitation of the polymer upon contact causes the polymer to form nanoparticles while the first solution is poured into the second liquid, and in some cases, for example, when the rate of introduction is carefully controlled and kept at a relatively slow rate, nanoparticles may form. The control of such particle formation can be readily optimized by one of ordinary skill in the art using only routine experimentation.

Properties such as surface functionality, surface charge, size, zeta (0 potential, hydrophobicity, ability to control immunogenicity, and the like, may be highly controlled using a disclosed process. For instance, a library of particles may be synthesized, and screened to identify the particles having a particular ratio of polymers that allows the particles to have a specific density of moieties (e.g., low-molecular weight ligands) present on the surface of the particle. This allows particles having one or more specific properties to be prepared, for example, a specific size and a specific surface density of moieties, without an undue degree of effort. Accordingly, certain embodiments are directed to screening techniques using such libraries, as well as any particles identified using such libraries. In addition, identification may occur by any suitable method. For instance, the identification may be direct or indirect, or proceed quantitatively or qualitatively.

In some embodiments, already-formed nanoparticles are functionalized with a targeting moiety using procedures analogous to those described for producing ligand-functionalized polymeric conjugates. For example, a first copolymer (PLGA-PEG, poly(lactide-co-glycolide) and poly(ethylene glycol)) is mixed with the therapeutic agent to form particles. The particles are then associated with a low-molecular weight ligand to form nanoparticles that can be used for the treatment of cancer. The particles can be associated with varying amounts of low-molecular weight ligands in order to control the ligand surface density of the nanoparticle, thereby altering the therapeutic characteristics of the nanoparticle. Furthermore, for example, by controlling parameters such as molecular weight, the molecular weight of PEG, and the nanoparticle surface charge, very precisely controlled particles may be obtained.

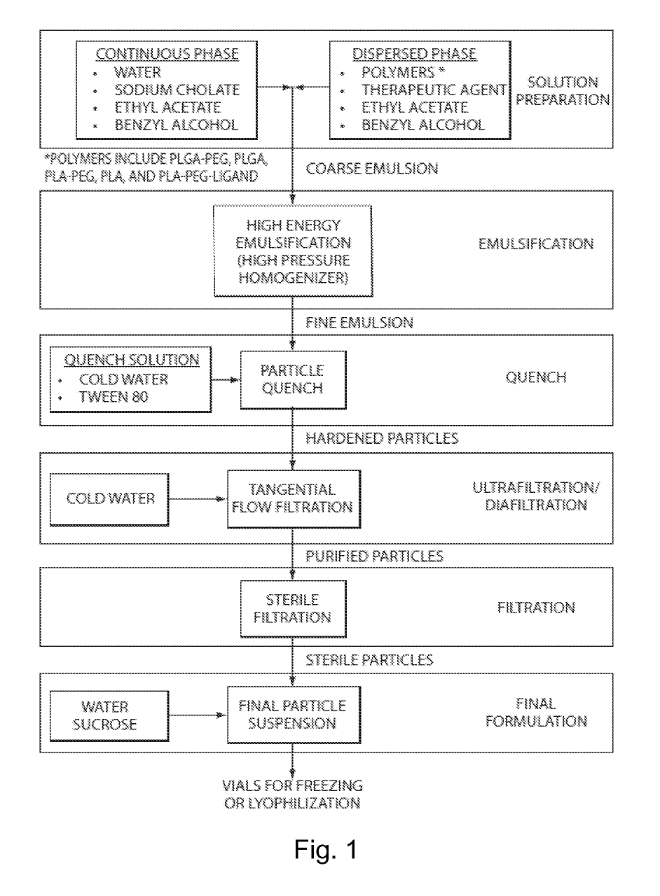

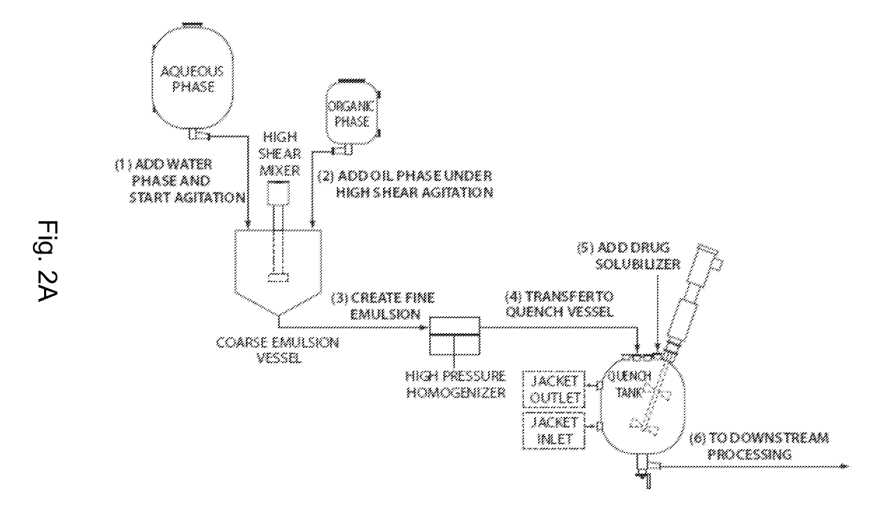

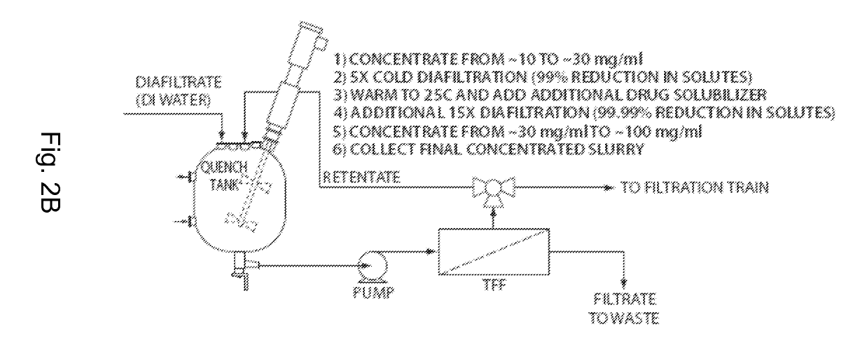

In another embodiment, a nanoemulsion process is provided, such as the process represented in FIGS. 1, 2A, and 2B. For example, a therapeutic agent (e.g., docetaxel), a hydrophobic acid, a first polymer (for example, a diblock co-polymer such as PLA-PEG or PLGA-PEG, either of which may be optionally bound to a ligand) and an optional second polymer (e.g., (PL(G)A-PEG or PLA), may be combined with an organic solution to form a first organic phase. Such first phase may include about 1 to about 50% weight solids, about 5 to about 50% weight solids, about 5 to about 40% weight solids, about 1 to about 15% weight solids, or about 10 to about 30% weight solids. The first organic phase may be combined with a first aqueous solution to form a second phase. The organic solution can include, for example, toluene, methyl ethyl ketone, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80, or the like, and combinations thereof. In an embodiment, the organic phase may include benzyl alcohol, ethyl acetate, and combinations thereof. The second phase can be between about 0.1 and 50 weight %, between about 1 and 50 weight %, between about 5 and 40 weight %, or between about 1 and 15 weight %, solids. The aqueous solution can be water, optionally in combination with one or more of sodium cholate, ethyl acetate, polyvinyl acetate and benzyl alcohol. In some embodiments, the pH of the aqueous phase may be selected based on the pKₐ of the therapeutic agent and/or the pKₐ of the hydrophobic acid or protonated hydrophobic base. For example, in certain embodiments, the therapeutic agent may have a first pKₐ, the hydrophobic acid or protonated hydrophobic base may have a second pKₐ, and the aqueous phase may have a pH equal to a pKₐ unit between the first pKₐ and the second pKₐ. In a particular embodiment, the pH of the aqueous phase may be equal to a pKₐ unit that is about equidistant between the first pKₐ and the second pKₐ.

For example, the oil or organic phase may use a solvent that is only partially miscible with the nonsolvent (water). Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol. In some instances, the organic phase (e.g., first organic phase) may include the basic therapeutic agent. Additionally, in certain embodiments, the aqueous solution (e.g., first aqueous solution) may include the substantially hydrophobic acid. In other embodiments, both the basic therapeutic agent and the substantially hydrophobic acid may be dissolved in the organic phase.

Emulsifying the second phase to form an emulsion phase may be performed, for example, in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g., probe sonicator or a high pressure homogenizer, e.g., by using 1, 2, 3, or more passes through a homogenizer. For example, when a high pressure homogenizer is used, the pressure used may be about 30 to about 60 psi, about 40 to about 50 psi, about 1000 to about 8000 psi, about 2000 to about 4000 psi, about 4000 to about 8000 psi, or about 4000 to about 5000 psi, e.g., about 2000, 2500, 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. In some embodiments, quenching may be performed at least partially at a temperature of about 5° C. or less. For example, water used in the quenching may be at a temperature that is less than room temperature (e.g., about 0 to about 10° C., or about 0 to about 5° C.).

In certain embodiments, the quench may be chosen having a pH that is advantageous for quenching the emulsion phase, e.g., by improving the properties of the nanoparticles, such as the release profile, or improving a nanoparticle parameter, such as the drug loading. The pH of the quench may be adjusted by acid or base titration, for example, or by appropriate selection of a buffer.

In some embodiments, the pH of the quench may be selected based on the pKₐ of the therapeutic agent and/or the pKₐ of the hydrophobic acid or protonated hydrophobic base. For example, in certain embodiments, the therapeutic agent may have a first pKₐ, the hydrophobic acid or protonated hydrophobic base may have a second pKₐ, and the emulsion phase may be quenched with an aqueous solution having a pH equal to a pKₐ unit between the first pKₐ and the second pKₐ. In some embodiments, the resultant quenched phase may also have a pH equal to a pKₐ unit between the first pKₐ and the second pKₐ. In a particular embodiment, the pH may be equal to a pKₐ unit that is about equidistant between the first pKₐ and the second pKₐ.

In some embodiments, the quench may have a pH between about 2 and about 12, in some embodiments between about 3 and about 10, in some embodiments between about 3 and about 9, in some embodiments between about 3 and about 8, in some embodiments between about 3 and about 7, in some embodiments between about 4 and about 8, in some embodiments between about 4 and about 7, in some embodiments between about 4 and about 6, in some embodiments between about 4 and about 5, in some embodiments between about 4.2 and about 4.8, in some embodiments between about 6 and about 10, in some embodiments between about 6 and about 9, in some embodiments between about 6 and about 8, in some embodiments between about 6 and about 7. In certain embodiments, the quench may have a pH of about 4.5. It should be understood that the pH of a buffer solution may vary as a function of temperature. Unless otherwise specified, the pH of a buffer solution referred to herein is the pH at 23° C.

In some embodiments, not all of the therapeutic agent is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, sodium cholate, diethylnitrosamine, sodium acetate, urea, glycerin, propylene glycol, glycofurol, poly(ethylene)glycol, bris(polyoxyethyleneglycolddodecyl ether, sodium benzoate, sodium salicylate, or combinations thereof. For example, Tween-80 may be added to the quenched nanoparticle suspension to solubilize the free drug and prevent the formation of drug crystals. In some embodiments, a ratio of drug solubilizer to the therapeutic agent is about 200:1 to about 10:1, or in some embodiments about 100:1 to about 10:1.

The solubilized phase may be filtered to recover the nanoparticles. For example, ultrafiltration membranes may be used to concentrate the nanoparticle suspension and substantially eliminate organic solvent, free drug (i.e., unencapsulated therapeutic agent), drug solubilizer, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (˜5-25 nm) may be used.

Diafiltration may be performed using a constant volume approach, meaning the diafiltrate (cold deionized water, e.g., about 0 to about 5° C., or 0 to about 10° C.) may added to the feed suspension at the same rate as the filtrate is removed from the suspension. In some embodiments, filtering may include a first filtering using a first temperature of about 0 to about 5° C., or 0 to about 10° C., and a second temperature of about 20 to about 30° C., or 15 to about 35° C. In some embodiments, filtering may include processing about 1 to about 30, in some cases about 1 to about 15, or in some cases 1 to about 6 diavolumes. For example, filtering may include processing about 1 to about 30, or in some cases about 1 to about 6 diavolumes, at about 0 to about 5° C., and processing at least one diavolume (e.g., about 1 to about 15, about 1 to about 3, or about 1 to about 2 diavolumes) at about 20 to about 30° C. In some embodiments, filtering comprises processing different diavolumes at different distinct temperatures.

After purifying and concentrating the nanoparticle suspension, the particles may be passed through one, two or more sterilizing and/or depth filters, for example, using ˜0.2 μm depth pre-filter. For example, a sterile filtration step may involve filtering the therapeutic nanoparticles using a filtration train at a controlled rate. In some embodiments, the filtration train may include a depth filter and a sterile filter.

### Therapeutic Agents

Disclosed nanoparticles may include a therapeutic agent such as an antineoplastic agent, e.g. such as a mTor inhibitor (e.g., sirolimus, temsirolimus, or everolimus), a vinca alkaloid such as vincristine, a diterpene derivative or a taxane such as paclitaxel (or its derivatives such as DHA-paclitaxel or PG-paclitaxel) or docetaxel.

In one set of embodiments, a disclosed nanoparticle may include a drug or a combination of more than one drug. Such particles may be useful, for example, in embodiments where a targeting moiety may be used to direct a particle containing a drug to a particular localized location within a subject, e.g., to allow localized delivery of the drug to occur. Exemplary therapeutic agents include chemotherapeutic agents such as doxorubicin (Adriamycin), gemcitabine (Gemzar), daunorubicin, procarbazine, mitomycin, cytarabine, etoposide, methotrexate, vinorelbine, 5-fluorouracil (5-FU), vinca alkaloids such as vinblastine or vincristine; bleomycin, paclitaxel (taxol), docetaxel (taxotere), aldesleukin, asparaginase, carboplatin, cladribine, camptothecin, 10-hydroxy-7-ethylcamptothecin (SN38), dacarbazine, S-I capecitabine, 5′deoxyflurouridine, eniluracil, deoxycytidine, 5-azacytosine, 5-azadeoxycytosine, allopurinol, 2-chloroadenosine, trimetrexate, aminopterin, methylene-10-deazaaminopterin (MDAM), oxaplatin, picoplatin, ormaplatin, epirubicin, etoposide phosphate, 9-aminocamptothecin, 10,11-methylenedioxycamptothecin, karenitecin, 9-nitrocamptothecin, vindesine, L-phenylalanine mustard, ifosphamidemefosphamide, perfosfamide, trophosphamide carmustine, semustine, epothilones A, B, C, D, and E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, and combinations thereof. Non-limiting examples of potentially suitable drugs include anti-cancer agents, including, for example, docetaxel, mitoxantrone, and mitoxantrone hydrochloride.

### Pharmaceutical Formulations

Nanoparticles disclosed herein may be combined with pharmaceutically acceptable carriers to form a pharmaceutical composition, according to another aspect. As would be appreciated by one of skill in this art, the carriers may be chosen based on the route of administration as described below, the location of the target issue, the drug being delivered, the time course of delivery of the drug, etc. In certain embodiments, the nanoparticles described herein may be formulated in a pharmaceutical composition for use in the therapeutic methods, such as treating cancer (e.g., cancer having a Ras mutation or a refractory cancer that is refractory to chemotherapy and/or radiation therapy alone).

The pharmaceutical compositions can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

It will be appreciated that the exact dosage of a nanoparticle containing a therapeutic agent is chosen by the individual physician in view of the patient to be treated, in general, dosage and administration are adjusted to provide an effective amount of the therapeutic agent nanoparticle to the patient being treated. As used herein, the “effective amount” of a nanoparticle containing a therapeutic agent refers to the amount necessary to elicit the desired biological response. As will be appreciated by those of ordinary skill in this art, the effective amount of a nanoparticle containing a therapeutic agent may vary depending on such factors as the desired biological endpoint, the drug to be delivered, the target tissue, the route of administration, etc. For example, the effective amount of a nanoparticle containing a therapeutic agent might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The nanoparticles may be formulated in dosage unit form for ease of administration and uniformity of dosage. The expression “dosage unit form” as used herein refers to a physically discrete unit of nanoparticle appropriate for the patient to be treated. It will be understood, however, that the total daily usage of the compositions will be decided by the attending physician within the scope of sound medical judgment. For any nanoparticle, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In an embodiment, compositions disclosed herein may include less than about 10 ppm of palladium, or less than about 8 ppm, or less than about 6 ppm of palladium. For example, provided here is a composition that includes nanoparticles having a polymeric conjugate wherein the composition has less than about 10 ppm of palladium.

In some embodiments, a composition suitable for freezing is contemplated, including nanoparticles disclosed herein and a solution suitable for freezing, e.g., a sugar such as a mono, di, or poly saccharide, e.g., sucrose and/or a trehalose, and/or a salt and/or a cyclodextrin solution is added to the nanoparticle suspension. The sugar (e.g., sucrose or trehalose) may act, e.g., as a cryoprotectant to prevent the particles from aggregating upon freezing. For example, provided herein is a nanoparticle formulation comprising a plurality of disclosed nanoparticles, sucrose, an ionic halide, and water; wherein the nanoparticles/sucrose/water/ionic halide is about 3-40%/10-40%/20-95%/0.1-10% (w/w/w/w) or about 5-10%/10-15%/80-90%/1-10% (w/w/w/w). For example, such solution may include nanoparticles as disclosed herein, about 5% to about 20% by weight sucrose and an ionic halide such as sodium chloride, in a concentration of about 10-100 mM. In another example, provided herein is a nanoparticle formulation comprising a plurality of disclosed nanoparticles, trehalose, cyclodextrin, and water; wherein the nanoparticles/trehalose/water/cyclodextrin is about 3-40%/1-25%/20-95%/1-25% (w/w/w/w) or about 5-10%/1-25%/80-90%/10-15% (w/w/w/w).

For example, a contemplated solution may include nanoparticles as disclosed herein, about 1% to about 25% by weight of a disaccharide such as trehalose or sucrose (e.g., about 5% to about 25% trehalose or sucrose, e.g. about 10% trehalose or sucrose, or about 15% trehalose or sucrose, e.g. about 5% sucrose) by weight) and a cyclodextrin such as β-cyclodextrin, in a concentration of about 1% to about 25% by weight (e.g. about 5% to about 20%, e.g. 10% or about 20% by weight, or about 15% to about 20% by weight cyclodextrin). Contemplated formulations may include a plurality of disclosed nanoparticles (e.g. nanoparticles having PLA-PEG and an active agent), and about 2% to about 15 wt % (or about 4% to about 6 wt %, e.g. about 5 wt %) sucrose and about 5 wt % to about 20% (e.g. about 7% wt percent to about 12 wt %, e.g. about 10 wt %) of a cyclodextrin, e.g., HPbCD).

The present disclosure relates in part to lyophilized pharmaceutical compositions that, when reconstituted, have a minimal amount of large aggregates. Such large aggregates may have a size greater than about 0.5 μm, greater than about 1 μm, or greater than about 10 μm, and can be undesirable in a reconstituted solution. Aggregate sizes can be measured using a variety of techniques including those indicated in the U.S. Pharmacopeia at 32 <788>, hereby incorporated by reference. The tests outlined in USP 32 <788> include a light obscuration particle count test, microscopic particle count test, laser diffraction, and single particle optical sensing. In one embodiment, the particle size in a given sample is measured using laser diffraction and/or single particle optical sensing.

The USP 32 <788> by light obscuration particle count test sets forth guidelines for sampling particle sizes in a suspension. For solutions with less than or equal to 100 mL, the preparation complies with the test if the average number of particles present does not exceed 6000 per container that are ≧10 μm and 600 per container that are ≧25 μm.

As outlined in USP 32 <788>, the microscopic particle count test sets forth guidelines for determining particle amounts using a binocular microscope adjusted to 100±10× magnification having an ocular micrometer. An ocular micrometer is a circular diameter graticule that consists of a circle divided into quadrants with black reference circles denoting 10 μm and 25 μm when viewed at 100× magnification. A linear scale is provided below the graticule. The number of particles with reference to 10 μm and 25 μm are visually tallied. For solutions with less than or equal to 100 mL, the preparation complies with the test if the average number of particles present does not exceed 3000 per container that are ≧10 μm and 300 per container that are ≧25 μm.

In some embodiments, a 10 mL aqueous sample of a disclosed composition upon reconstitution comprises less than 600 particles per ml having a size greater than or equal to 10 microns; and/or less than 60 particles per ml having a size greater than or equal to 25 microns.

Dynamic light scattering (DLS) may be used to measure particle size, but it relies on Brownian motion so the technique may not detect some larger particles. Laser diffraction relies on differences in the index of refraction between the particle and the suspension media. The technique is capable of detecting particles at the sub-micron to millimeter range. Relatively small (e.g., about 1-5 weight %) amounts of larger particles can be determined in nanoparticle suspensions. Single particle optical sensing (SPOS) uses light obscuration of dilute suspensions to count individual particles of about 0.5 μm. By knowing the particle concentration of the measured sample, the weight percentage of aggregates or the aggregate concentration (particles/mL) can be calculated.

Formation of aggregates can occur during lyophilization due to the dehydration of the surface of the particles. This dehydration can be avoided by using lyoprotectants, such as disaccharides, in the suspension before lyophilization. Suitable disaccharides include sucrose, lactulose, lactose, maltose, trehalose, or cellobiose, and/or mixtures thereof. Other contemplated disaccharides include kojibiose, nigerose, isomaltose, β,β-trehalose, α,β-trehalose, sophorose, laminaribiose, gentiobiose, turanose, maltulose, palatinose, gentiobiulose, mannobiase, melibiose, melibiulose, rutinose, rutinulose, and xylobiose. Reconstitution shows equivalent DLS size distributions when compared to the starting suspension. However, laser diffraction can detect particles of >10 μm in size in some reconstituted solutions. Further, SPOS also may detect >10 μm sized particles at a concentration above that of the FDA guidelines (10⁴-10⁵ particles/mL for >10 μm particles).

In some embodiments, one or more ionic halide salts may be used as an additional lyoprotectant to a sugar, such as sucrose, trehalose or mixtures thereof. Sugars may include disaccharides, monosaccharides, trisaccharides, and/or polysaccharides, and may include other excipients, e.g. glycerol and/or surfactants. Optionally, a cyclodextrin may be included as an additional lyoprotectant. The cyclodextrin may be added in place of the ionic halide salt. Alternatively, the cyclodextrin may be added in addition to the ionic halide salt.

Suitable ionic halide salts may include sodium chloride, calcium chloride, zinc chloride, or mixtures thereof. Additional suitable ionic halide salts include potassium chloride, magnesium chloride, ammonium chloride, sodium bromide, calcium bromide, zinc bromide, potassium bromide, magnesium bromide, ammonium bromide, sodium iodide, calcium iodide, zinc iodide, potassium iodide, magnesium iodide, or ammonium iodide, and/or mixtures thereof. In one embodiment, about 1 to about 15 weight percent sucrose may be used with an ionic halide salt. In one embodiment, the lyophilized pharmaceutical composition may comprise about 10 to about 100 mM sodium chloride. In another embodiment, the lyophilized pharmaceutical composition may comprise about 100 to about 500 mM of divalent ionic chloride salt, such as calcium chloride or zinc chloride. In yet another embodiment, the suspension to be lyophilized may further comprise a cyclodextrin, for example, about 1 to about 25 weight percent of cyclodextrin may be used.

A suitable cyclodextrin may include α-cyclodextrin, β-cyclodextrin, γ-cyclodextrin, or mixtures thereof. Exemplary cyclodextrins contemplated for use in the compositions disclosed herein include hydroxypropyl-β-cyclodextrin (HPbCD), hydroxyethyl-β-cyclodextrin, sulfobutylether-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-β-cyclodextrin, glucosyl-β-cyclodextrin, and maltosyl-β-cyclodextrin. In one embodiment, about 1 to about 25 weight percent trehalose (e.g. about 10% to about 15%, e.g. 5 to about 20% by weight) may be used with cyclodextrin. In one embodiment, the lyophilized pharmaceutical composition may comprise about 1 to about 25 weight percent β-cyclodextrin. An exemplary composition may comprise nanoparticles comprising PLA-PEG, an active/therapeutic agent, about 4% to about 6% (e.g. about 5% wt percent) sucrose, and about 8 to about 12 weight percent (e.g. about 10 wt. %) HPbCD.

In one aspect, a lyophilized pharmaceutical composition is provided comprising disclosed nanoparticles, wherein upon reconstitution of the lyophilized pharmaceutical composition at a nanoparticle concentration of about 50 mg/mL, in less than or about 100 mL of an aqueous medium, the reconstituted composition suitable for parenteral administration comprises less than 6000, such as less than 3000, microparticles of greater than or equal to 10 microns; and/or less than 600, such as less than 300, microparticles of greater than or equal to 25 microns.

The number of microparticles can be determined by means such as the USP 32 <788> by light obscuration particle count test, the USP 32 <788> by microscopic particle count test, laser diffraction, and single particle optical sensing.

In an aspect, a pharmaceutical composition suitable for parenteral use upon reconstitution is provided comprising a plurality of therapeutic particles each comprising a copolymer having a hydrophobic polymer segment and a hydrophilic polymer segment; an active agent; a sugar; and a cyclodextrin.

For example, the copolymer may be poly(lactic) acid-block-poly(ethylene)glycol copolymer. Upon reconstitution, a 100 mL aqueous sample may comprise less than 6000 particles having a size greater than or equal to 10 microns; and less than 600 particles having a size greater than or equal to 25 microns.

The step of adding a disaccharide and an ionic halide salt may comprise adding about 5 to about 15 weight percent sucrose or about 5 to about 20 weight percent trehalose (e.g., about 10 to about 20 weight percent trehalose), and about 10 to about 500 mM ionic halide salt. The ionic halide salt may be selected from sodium chloride, calcium chloride, and zinc chloride, or mixtures thereof. In an embodiment, about 1 to about 25 weight percent cyclodextrin is also added.

In another embodiment, the step of adding a disaccharide and a cyclodextrin may comprise adding about 5 to about 15 weight percent sucrose or about 5 to about 20 weight percent trehalose (e.g., about 10 to about 20 weight percent trehalose), and about 1 to about 25 weight percent cyclodextrin. In an embodiment, about 10 to about 15 weight percent cyclodextrin is added. The cyclodextrin may be selected from α-cyclodextrin, β-cyclodextrin, γ-cyclodextrin, or mixtures thereof.

In another aspect, a method of preventing substantial aggregation of particles in a pharmaceutical nanoparticle composition is provided comprising adding a sugar and a salt to the lyophilized formulation to prevent aggregation of the nanoparticles upon reconstitution. In an embodiment, a cyclodextrin is also added to the lyophilized formulation. In yet another aspect, a method of preventing substantial aggregation of particles in a pharmaceutical nanoparticle composition is provided comprising adding a sugar and a cyclodextrin to the lyophilized formulation to prevent aggregation of the nanoparticles upon reconstitution.

A contemplated lyophilized composition may have a therapeutic particle concentration of greater than about 40 mg/mL. The formulation suitable for parenteral administration may have less than about 600 particles having a size greater than 10 microns in a 10 mL dose. Lyophilizing may comprise freezing the composition at a temperature of greater than about −40° C., or e.g. less than about −30° C., forming a frozen composition; and drying the frozen composition to form the lyophilized composition. The step of drying may occur at about 50 mTorr at a temperature of about −25 to about −34° C., or about −30 to about −34° C.

### EXAMPLES

The invention now being generally described, it will be more readily understood by reference to the following examples which are included merely for purposes of illustration of certain aspects and embodiments of the present invention, and are not intended to limit the invention in any way.

### Example 1: Nanoparticle Preparation—Emulsion Process

An organic phase is formed composed of a mixture of docetaxel (DTXL) and polymer (co-polymer, and/or co-polymer with ligand). The organic phase is mixed with an aqueous phase at approximately a 1:5 ratio (oil phase:aqueous phase) where the aqueous phase is composed of a surfactant and some dissolved solvent. In order to achieve high drug loading, about 30% solids in the organic phase is used.

The primary, coarse emulsion is formed by the combination of the two phases under simple mixing or through the use of a rotor stator homogenizer. The rotor/stator yielded a homogeneous milky solution, while the stir bar produced a visibly larger coarse emulsion. It was observed that the stir bar method resulted in significant oil phase droplets adhering to the side of the feed vessel, suggesting that while the coarse emulsion size is not a process parameter critical to quality, it should be made suitably fine in order to prevent yield loss or phase separation. Therefore the rotor stator is used as the standard method of coarse emulsion formation, although a high speed mixer may be suitable at a larger scale.

The primary emulsion is then formed into a fine emulsion through the use of a high pressure homogenizer. The size of the coarse emulsion does not significantly affect the particle size after successive passes (103) through the homogenizer M-110-EH.

Homogenizer feed pressure was found to have a significant impact on resultant particle size. On both the pneumatic and electric M-110EH homogenizers, it was found that reducing the feed pressure also reduced the particle size. Therefore the standard operating pressure used for the M-110EH is 4000-5000 psi per interaction chamber, which is the minimum processing pressure on the unit. The M-110EH also has the option of one or two interaction chambers. It comes standard with a restrictive Y-chamber, in series with a less restrictive 200 μm Z-chamber. It was found that the particle size was actually reduced when the Y-chamber was removed and replaced with a blank chamber. Furthermore, removing the Y-chamber significantly increases the flow rate of emulsion during processing.

After 2-3 passes the particle size was not significantly reduced, and successive passes can even cause a particle size increase. Table A summarizes the emulsification process parameters.

TABLE A

| Parameter | Value | Observation |
|---|---|---|
| Coarse emulsion | Rotor stator | Coarse emulsion size does not affect final particle size, but large |
| formation | homogenizer | coarse emulsion can cause increased oil phase retention in feed vessel |
| Homogenizer feed | 4000-5000 psi per | Lower pressure reduces particle size |
| pressure | chamber | |
| Interaction | 2 × 200 μm Z- | 200 μm Z-chamber yields the smallest particle size, and allows for |
| chamber(s) | chamber | highest homogenizer throughput |
| Number of | 2-3 passes | Studies have shown that the particle size is not significantly reduced |
| homogenizer passes | | after 2 discreet passes, and size can even increase with successive |
| | | passes |
| Water phase | 0.1% | [Sodium cholate] can effectively alter particle size; value is optimized |
| [sodium cholate] | | for given process and formulation |
| W:O ratio | 5:1 | Lowest ratio without significant particle size increase is ~5:1 |
| [Solids] in oil phase | 30% | Increased process efficiency, increased drug encapsulation, workable |
| | | viscosity |

The fine emulsion is then quenched by addition to deionized water at a given temperature under mixing. In the quench unit operation, the emulsion is added to a cold aqueous quench under agitation. This serves to extract a significant portion of the oil phase solvents, effectively hardening the nanoparticles for downstream filtration. Chilling the quench significantly improved drug encapsulation. The quench:emulsion ratio is approximately 5:1.

A solution of 35% (wt %) of Tween 80 is added to the quench to achieve approximately 2% Tween 80 overall After the emulsion is quenched a solution of Tween-80 is added which acts as a drug solubilizer, allowing for effective removal of unencapsulated drug during filtration. Table B indicates each of the quench process parameters.

TABLE B
Summary quench process parameters.

| Parameter | Value | Observation |
|---|---|---|
| Initial quench | <5° C. | Low temperature yields higher |
| temperature | | drug encapsulation |
| [Tween-80] | 35% | Highest concentration that |
| solution | | can be prepared and readily |
| | | disperses in quench |
| Tween-80:drug | 25:1 | Minimum amount of Tween-80 |
| ratio | | required to effectively remove |
| | | unencapsulated drug |
| Q:E ratio | 5:1 | Minimum Q:E ratio while retain- |
| | | ing high drug encapsulation |
| Quench hold/ | ≦5° C. (with | Temperature which prevents |
| processing temp | current 5:1 Q:E | significant drug leaching |
| | ratio, 25:1 Tween- | during quench hold time and |
| | 80:drug ratio) | initial concentration step |

The temperature must remain cold enough with a dilute enough suspension (low enough concentration of solvents) to remain below the T_{g} of the particles. If the Q:E ratio is not high enough, then the higher concentration of solvent plasticizes the particles and allows for drug leakage. Conversely, colder temperatures allow for high drug encapsulation at low Q:E ratios (to ˜3:1), making it possible to run the process more efficiently.

The nanoparticles are then isolated through a tangential flow filtration process to concentrate the nanoparticle suspension and buffer exchange the solvents, free drug, and drug solubilizer from the quench solution into water. A regenerated cellulose membrane is used with a molecular weight cutoffs (MWCO) of 300.

A constant volume diafiltration (DF) is performed to remove the quench solvents, free drug and Tween-80. To perform a constant-volume DF, buffer is added to the retentate vessel at the same rate the filtrate is removed. The process parameters for the TFF operations are summarized in Table C. Crossflow rate refers to the rate of the solution flow through the feed channels and across the membrane. This flow provides the force to sweep away molecules that can foul the membrane and restrict filtrate flow. The transmembrane pressure is the force that drives the permeable molecules through the membrane.

TABLE C
TFF Parameters

| | Optimized | | |
|---|---|---|---|
| Parameter | Value | | Effect |
| Membrane | Regenerated | | No difference in performance between RC and PES, but |
| Material | cellulose - | | solvent compatibility is superior for RC. |
| | Coarse Screen | | |
| | Membrane | | |
| Molecular Weight | 300 | kDa | No difference in NP characteristics (i.e. residual |
| Cut off | | | tween)Increase in flux rates is seen with 500 kDa |
| | | | membrane but 500 kDa is not available in RC |
| Crossflow Rate | 11 | L/min/m² | Higher crossflow rate led to higher flux |
| Transmembrane | 20 | psid | Open channel membranes have maximum flux rates |
| Pressure | | | between 10 and 30 psid. Coarse channel membranes have |
| | | | maximum flux rates with min TMP (~20 psid). |
| Concentration of | 30 | mg/ml | Diafiltration is most efficient at [NP] ~50 mg/ml with |
| Nanoparticle | | | open channel TFF membranes based on flux rates and |
| Suspension for | | | throughput. With coarse channel membranes the flux rate |
| Diafiltration | | | is optimized at ~30 mg/ml in the starting buffer. |
| Number of | ≧15 (based on | | About 15 diavolumes are needed to effectively remove |
| Diavolumes | flux increase) | | tween-80. End point of diafiltration is determined by in- |
| | | | process control (flux increase plateau). |
| Membrane Area | ~1 | m²/kg | Membranes sized based on anticipated flux rates and |
| | | | volumes required. |

The filtered nanoparticle slurry is then thermal cycled to an elevated temperature during workup. A small portion (typically 5-10%) of the encapsulated drug is released from the nanoparticles very quickly after its first exposure to 25° C. Because of this phenomenon, batches that are held cold during the entire workup are susceptible to free drug or drug crystals forming during delivery or any portion of unfrozen storage. By exposing the nanoparticle slurry to elevated temperature during workup, this ‘loosely encapsulated’ drug can be removed and improve the product stability at the expense of a small drop in drug loading. Table D summarizes two examples of 25° C. processing. Other experiments have shown that the product is stable enough after ˜2-4 diavolumes to expose it to 25° C. without losing the majority of the encapsulated drug. 5 diavolumes is used as the amount for cold processing prior to the 25° C. treatment.

TABLE D

| | | Lots A | Lots B |
|---|---|---|---|
| Drug load | Cold workup | 11.3% | 9.7% |
| | 25° C. workup¹ | 8.7-9.1% | 9.0-9.9% |
| Stability² | Cold workup | <1 day | <1 day |
| | 25° C. workup¹ | 5-7 days | 2-7 days |
| In vitro burst³ | Cold workup | ~10% | Not |
| | 25° C. workup¹ | ~2% | performed |

¹25° C. workup sublots were exposed to 25° C. after at least 5 diavolumes for various periods of time. Ranges are reported because there were multiple sublots with 25° C. exposure.
²Stability data represents the time that final product could be held at 25° C. at 10-50 mg/ml nanoparticle concentrations prior to crystals forming in the slurry (visible by microscopy)
³In vitro burst represents the drug released at the first time point (essentially immediately)

After the filtration process the nanoparticle suspension is passed through a sterilizing grade filter (0.2 μm absolute). Pre-filters are used to protect the sterilizing grade filter in order to use a reasonable filtration area/time for the process. Values are as summarized in Table E.

TABLE E

| Parameter | O Value | | Effect |
|---|---|---|---|
| Nanoparticle | 50 | mg/ml | Yield losses are higher at higher |
| Suspension | | | [NP], but the ability to |
| Concentration | | | filter at 50 mg/ml obviates the |
| | | | need to aseptically concentrate |
| | | | after filtration |
| Filtration | ~1.3 | L/min/m² | Filterability decreases as flow |
| flow rate | | | rate increases |

The filtration train is Ertel Alsop Micromedia XL depth filter M953P membrane (0.2 μm Nominal); Pall SUPRAcap with Seitz EKSP depth filter media (0.1-0.3 μm Nominal); Pall Life Sciences Supor EKV 0.65/0.2 micron sterilizing grade PES filter.

0.2 m² of filtration surface area per kg of nanoparticles for depth filters and 1.3 m2 of filtration surface area per kg of nanoparticles for the sterilizing grade filters can be used.

### Example 2—Treatment of Human Non-Small Cell Lung Cancer

### Nanoparticle Composition

BIND-014: Docetaxel formulated in nanoparticles prepared as in Example 1 (10 wt % docetaxel, 90 wt % polymer (˜2.5 wt % PLA-PEG-GL2; and ˜97.5 wt % PLA-PEG, Mn PLA=16 kDa; Mn PEG=5 kDa) for intravenous injection.

### Study Population

Approximately 20 patients with Stage III/IV non-small cell lung cancer (NSCLC) who have failed one prior platinum-containing chemotherapy regimen for advanced or metastatic disease were enrolled from the United States and Russia. Of the 20 patients, 6 had known K-Ras mutations. K-Ras mutant lung cancer is associated with a worse prognosis than standard non-small cell lung cancer. Of these 6 patients, 2 progressed with disease, 2 patients had stable disease, and 2 patients had partial responses to the BIND-014 formulation.

### Study Drug, Dose, and Mode of Administration

Arm A: BIND-014 every 3 weeks. Patients received a 60 (±10) minute IV infusion of 60 mg/m² BIND-014 (in either 0.9% sodium chloride solution or 5% dextrose solution) administered once every three weeks (day 1 of a 21-day cycle). Patients also received a premedication regimen per protocol or institutional standards.

Arm B: BIND-014 weekly. Patients received a 60 (±10) minute IV infusion of 40 mg/m² BIND-014 (in either 0.9% sodium chloride solution or 5% dextrose solution) administered on a weekly schedule (days 1, 8 and 15 of a 28-day cycle). Patients also received a premedication regimen per protocol or institutional standards.

### Duration of Treatment

Screening (Baseline) Period. Patients had a screening period of 28 days prior to first treatment.

Treatment Period. Patients were treated until the disease progression as defined in the protocol or for other reasons of discontinuation as defined in the protocol.

Post-Treatment Follow-Up. Once a patient completed the treatment period, overall survival follow-up will be performed every 3 months for up to 60 months (5 years) in order to obtain post-study survival data and time to disease progression. Follow-up will be performed by telephone, clinical visit, or other documented contact per the institution's standard practice and will be reported on the case report form.

### Example 3—Treatment of Human Non-Small Cell Lung Cancer

### Nanoparticle Composition

BIND-014: Docetaxel formulated in nanoparticles prepared as in Example 1 (10 wt % docetaxel, 90 wt % polymer (˜2.5 wt % PLA-PEG-GL2; and ˜97.5 wt % PLA-PEG, Mn PLA=16 kDa; Mn PEG=5 kDa) for intravenous injection.

### Study Design

First stage: 20 patients; 1 responders out of 20 (CR or PR). Second stage: additional 20 patients.

Tumor data: Local reads were used for data analysis involving tumor activity. Determination of objective response was based on investigator-reviewed computed tomography (CT).

BIND-014 was administered at 60 mg/m² on day 1 of a 21-day cycle (i.e., one dose every 21-days) as measured by investigator assessed objective response rate (ORR) in patients with Stage III/W non-small cell lung cancer (NSCLC) who have failed one prior platinum containing chemotherapy regimen for advanced or metastatic disease.

### Study Population

Forty patients with Stage III/IV NSCLC who have failed one prior platinum-containing chemotherapy regimen for advanced or metastatic disease were enrolled.

Inclusion Criteria:
Diagnosis of NSCLC with locally advanced or metastatic disease
Known genomic status of disease at study entry. (EGFR mutation, ALK gene rearrangement, and K-Ras mutation)
Previously treated with one platinum-based chemotherapy
Disease status must be that of measurable and/or evaluable disease as defined by Response Evaluation Criteria in Solid Tumors (RECIST v. 1.1)
Performance status of 0 to 1 on the ECOG Scale
Prior chemotherapy/radiation completed at least 3 weeks prior to study enrollment
Prior radiation therapy allowed to <25% of the bone marrow
Adequate organ function

Exclusion Criteria:
Planning to become pregnant, pregnancy or breast feeding
Serious concomitant systemic disorders
Patients who are symptomatic from brain metastasis
Presence of detectable (by physical exam) third-space fluid collections
More than 1 prior cytotoxic chemotherapy regimen for advanced disease
Including switch maintenance chemotherapy (i.e. early second-line therapy)
Prior treatment with docetaxel
History of severe hypersensitivity reaction to polysorbate 80
Peripheral neuropathy at study entry
Congenital long QT syndrome, congestive heart failure, or bradyarrhythmia

Baseline patient demographics are provided in Table 1 below.

TABLE 1
Baseline Patient Demographics.
60 mg/m² of BIND-014 on day 1 of a 21-day cycle (N = 40)

| Sex | |
|---|---|
| Male/Female (%) | 73/27 |
| Age | |
| Median (years) | 62 |
| Range (years) | 40-85 |
| Performance Status (%) | |
| 0 | 42 |
| 1 | 58 |
| Stage IV (%) | 88 |
| Histology (%) | |
| Adenocarcinoma | 70 |
| Squamous cell carcinoma | 23 |
| Other/Undefined | 8 |
| Prior paclitaxel (%) | 15 |
| Best response, any prior chemo (%) | |
| CR/PR/SD | 55 |
| PD | 48 |
| Patients with disease free interval <3 | 55 |
| months on first line therapy | |
| Mutation Status (%) | |
| ALK Mutant | 0 |
| EGFR Mutant | 8 |
| K-Ras Mutant | 23 |

Of the 40 patients, 9 had known K-Ras mutations. K-Ras mutant lung cancer is associated with a worse prognosis than standard non-small cell lung cancer.

### Study Results

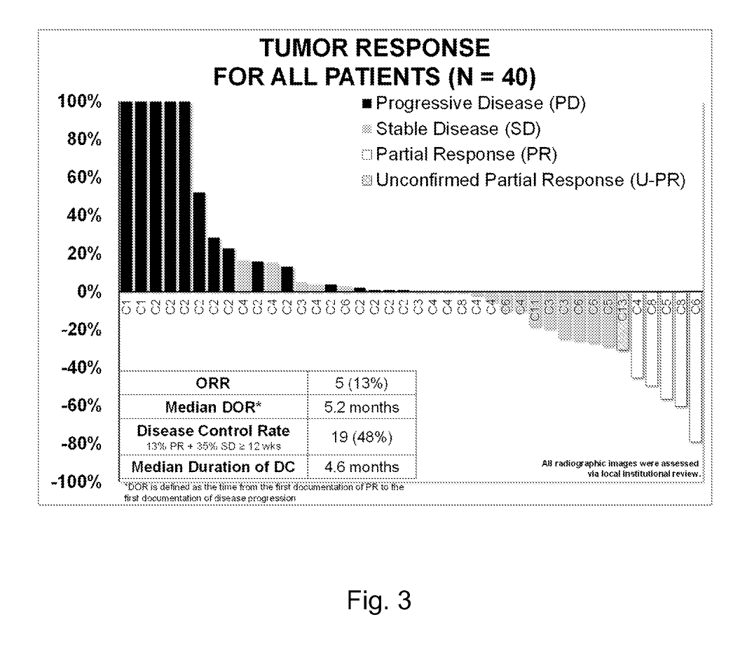

Tumor response for all patients (N=40) is shown in FIG. 3. Of the 40 patients, 15 progressed with disease, 19 patients had stable disease, 5 patients had partial responses to the BIND-014 formulation, and 1 patient had an unconfirmed partial response.

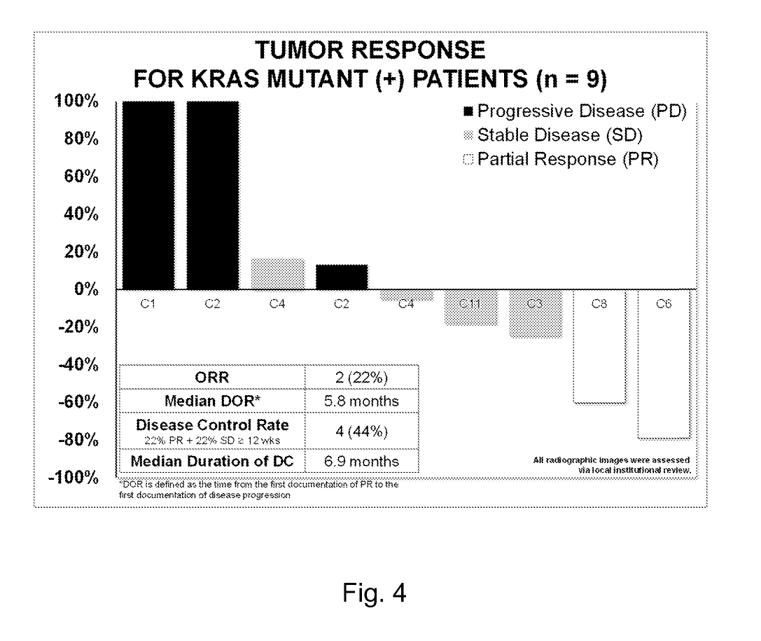

Tumor response for K-Ras mutant patients (N=9) is shown in FIG. 4. Of the 9 patients, 3 progressed with disease, 4 patients had stable disease, and 2 patients had partial responses to the BIND-014 formulation.

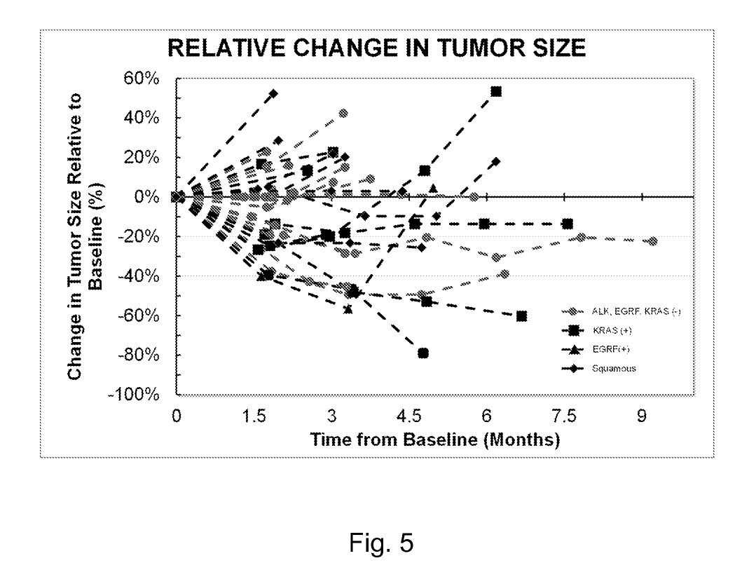

Relative change in tumor size as a function of time for all patients is shown in FIG. 5.

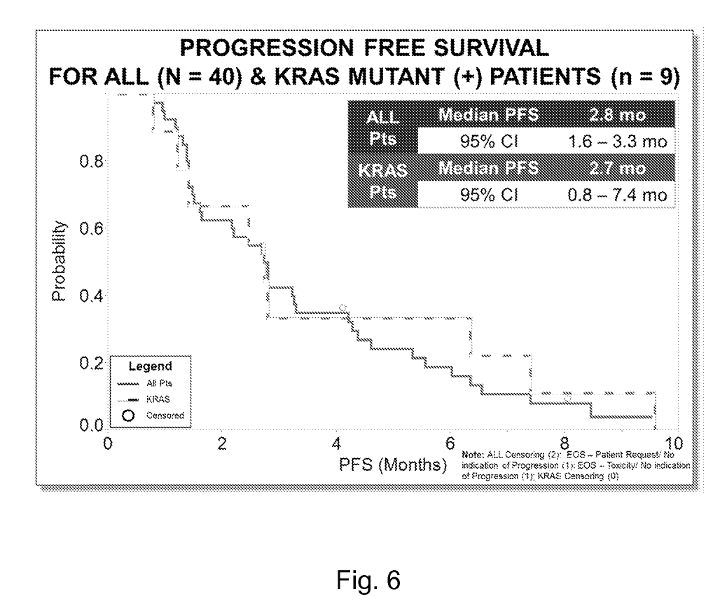

Progression free survival (PFS) for all patients (Pts) as compared to K-Ras mutant patients (Pts) is shown in FIG. 6.

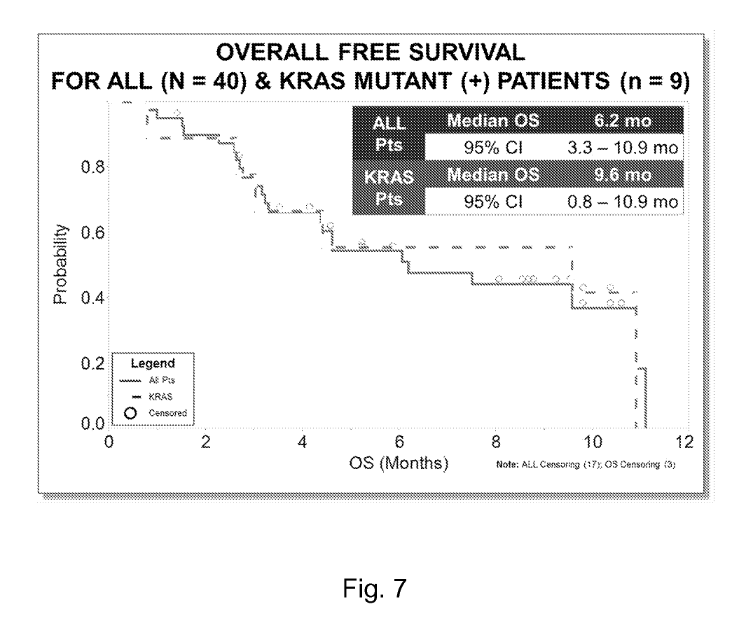

Overall free survival (OS) for all patients (Pts) as compared to K-Ras mutant patients (Pts) is shown in FIG. 7.

Treatment and response data are provided in Table 2 below.

TABLE 2
Treatment and Response Data.

| | All Patients | K-Ras Mutant Patients |
|---|---|---|
| | (N = 40) | (N = 9) |
| BIND-014 Treatment (n) | | |
| No. of cycles given, Median | 4 | 4 |
| No. of cycles given, Range | 1-13 | 1-11 |
| No. of patients receiving ≧4 | 21 | 5 |
| cycles | | |
| Off Treatment Reason (%) | | |
| Progression or Death | 75 | 67 |
| Toxicity | 10 | 0 |
| Other | 15 | 33 |
| Best Overall Response (%) | | |
| Partial Response | 13 | 22 |
| Stable Disease | 50 | 44 |
| Progressive Disease | 38 | 33 |

Treatment-related adverse event data for all patients (N=40) are provided in Table 3 below.

TABLE 3
Treatment-Related Adverse Event Data.*

| Event | Any Grade | Grade 3 & 4 |
|---|---|---|
| Hematologic | | |
| Anemia | 20 | 0 |
| Neutropenia | 10 | 5 |
| Lymphopenia | 5 | 3 |
| Infection | 10 | 0 |
| Febrile Neutropenia | 3 | 3 |
| Lymphopenia | 5 | 0 |
| Leukopenia | 3 | 0 |
| Non-Hematologic (%) | | |
| Fatigue | 33 | 5 |
| Nausea | 25 | 3 |
| Deceased Appetite | 18 | 0 |
| Hypersensitivity | 18 | 3 |
| Alopecia | 15 | 0 |
| Edema Peripheral | 15 | 0 |

*Treatment-related adverse events occurring in greater than or equal to 15% of patients.

The data show that BIND-014 is clinically active and well-tolerated at a dose of 60 mg/m² every 21 days as a second-line therapy in Stage III/IV NSCLC patients. BIND-014 demonstrated a 13% ORR with a median duration of response (DOR) of 5.2 months, and 48% DCR. In patients with K-Ras mutant tumors, which do not generally respond to known anti-cancer agents (e.g., Taxotere® and TKIs), BIND-014 demonstrated a 22% ORR with a median duration of response of 5.8 months, and 44% DCR.

Neutropenia, anemia, and neuropathy, commonly observed with Taxotere®, were significantly reduced with BIND-014.

Interim median overall survival for all patients treated was 6.2 months (95% CI: 3.3-10.9) (17 patients were censored). For patients with K-Ras mutant tumors, interim median survival was 9.6 months (95% CI: 0.8-10.9) (3 patients were censored).

The clinical activity and tolerability of BIND-014 indicate substantial differentiation from conventional docetaxel (Taxotere®). BIND-014 presents a novel option for patients with K-Ras mutant tumors.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A method of treating cancer having a Ras mutation in a patient in need thereof, comprising administering to the patient a therapeutically effective amount of a nanoparticle composition comprising anti-cancer nanoparticles, wherein the anti-cancer nanoparticles comprise a cyclodextrin.

2. The method of claim 1, wherein the anti-cancer nanoparticles are selected from the group consisting of polymeric nanoparticles, inorganic nanoparticles, metal nanoparticles, ceramic nanoparticles, nanocrystals, liposomes, micelles, and dendrimers.

3. The method of claim 1, wherein the anti-cancer nanoparticles are formed from a therapeutic agent.

4. The method of claim 2 claim 1 or 2, wherein the anti-cancer nanoparticles comprise a therapeutic agent.

5-7. (canceled)

8. The method of claim 3 any one of claims 3-7, wherein the therapeutically effective amount of the nanoparticle composition is about 50 to about 75 mg/m² of the therapeutic agent.

9-10. (canceled)

11. The method of claim 3 any one of claims 3-10, wherein the therapeutic agent is docetaxel.

12. The method of claim 1 any one of claims 1-11, further comprising administering the composition about every three weeks to said patient.

13. The method of claim 1 any one of claims 1-12, wherein the composition is administered by intravenous infusion over about 1 hour.

14. The method of claim 1 any one of claims 3-13, wherein the cancer was not stabilized by administration to the patient of free therapeutic agent.

15. The method of claim 1 any one of claims 1-14, wherein the hydrodynamic diameter of the nanoparticles is about 60 to about 150 nm.

16-17. (canceled)

18. The method of claim 1 any one of claims 1-17, wherein the nanoparticles comprise a diblock poly(lactic) acid-poly(ethylene) glycol copolymer or a diblock poly(lactic acid-co-glycolic acid)-poly(ethylene)glycol copolymer.

19. The method of claim 3 any one of claims 3-18, wherein the nanoparticles substantially retain the therapeutic agent for at least 1 minute when placed in a phosphate buffer solution at 37° C.

20-21. (canceled)

22. The method of claim 18 any one of claims 18-21, wherein the poly(lactic) acid-poly(ethylene)glycol copolymer has a poly(lactic) acid number average molecular weight fraction of about 0.6 to about 0.95.

23-25. (canceled)

26. The method of claim 18 any one of claims 18-26, wherein the nanoparticles comprise about 10 to about 25 weight percent poly(ethylene)glycol.

27-29. (canceled)

30. The method of claim 18 any one of claims 18-29, wherein the poly(lactic) acid-poly(ethylene)glycol copolymer has a number average molecular weight of about 15 kDa to about 20 kDa poly(lactic acid) and a number average molecular weight of about 4 kDa to about 6 kDa poly(ethylene)glycol.

31. The method of claim 1 any one of claims 1-30, wherein the nanoparticles further comprise about 0.2 to about 30 weight percent poly(lactic) acid-poly(ethylene)glycol copolymer functionalized with a targeting ligand.

32. The method of claim 1 any one of claims 1-31, wherein the nanoparticles further comprise about 0.2 to about 30 weight percent poly(lactic) acid-co-poly(glycolic) acid-poly(ethylene)glycol copolymer functionalized with a targeting ligand.

33-35. (canceled)

36. A method of treating a refractory cancer in a patient in need thereof, comprising administering to the patient a therapeutically effective amount of a nanoparticle composition comprising anti-cancer nanoparticles, wherein the refractory cancer is refractory to chemotherapy and/or radiation therapy alone, and wherein the anti-cancer nanoparticles comprise a cyclodextrin.

37-43. (canceled)

44. The method of claim 36 any one of claims 41-43, wherein the therapeutic agent is a taxane, a vinca alkaloid, or a mTor inhibitor.

45-61. (canceled)

62. The method of claim 1 any one of the above claims, wherein the nanoparticle composition is administered according to a treatment cycle.

63-74. (canceled)
